(19) United States
(12) Patent Application Publication
(10) Pub. No.: US 2026/0152532 A1
(43) Pub. Date: Jun. 4, 2026

(54) METHODS AND COMPOSITIONS COMPRISING FLT3L FOR THE TREATMENT OF WOUNDS

(71) Applicant: THE UNIVERSITY OF CHICAGO, Chicago, IL (US)

(72) Inventors: Jeffrey A. HUBBELL, Chicago, IL (US); Aaron T. ALPAR, Chicago, IL (US); Abigail L. LAUTERBACH, Chicago, IL (US); Kirsten C. REFVIK, Chicago, IL (US)

(21) Appl. No.: 19/123,495

(22) PCT Filed: Oct. 23, 2023
(86) PCT No.: PCT/US2023/077564

§ 371 Date: Apr. 23, 2025

Related U.S. Application Data
(60) Provisional application No. 63/418,725, filed on Oct. 24, 2022.

(51) Int. Cl. C07K 14/475; A61K 38/00; A61P 17/02

(52) U.S. Cl. CPC C07K 14/475; A61P 17/02; A61K 38/00; C07K 2319/30; C07K 2319/31

(57) ABSTRACT

Flt3L is a soluble, focused signal that can expand the dendritic cell compartment. The disclosure provides for methods for regenerating tissue, for preventing wound formation, or for treating wounds in a subject. The methods are achieved by administering a composition comprising a fms related tyrosine kinase 3 ligand (Flt3L) polypeptide to the subject.

### CROSS-REFERENCE TO RELATED APPLICATIONS

This application claims priority to U.S. Provisional Application Ser. No. 63/418,725, filed Oct. 24, 2022, which is incorporated herein in its entirety.

### SEQUENCE LISTING

The application contains a Sequence Listing prepared in compliance with ST.26 format and is hereby incorporated by reference in its entirety. Said Sequence Listing, created on Oct. 11, 2023 is named ARCDP0788WO.xml and is 71,563 bytes in size.

### BACKGROUND

### II. Field of the Invention

The invention generally relates to the field of medicine. More particularly, it concerns compositions and methods for treating wounds.

### III. Background

Wound healing is an important biological process that allows organisms to maintain homeostasis; without the potential to regenerate physiological barriers, one would be left vulnerable to infection. Chronic, non-healing wounds have reached epidemic proportions and have a sustainable impact on both an individual's quality of life and on healthcare costs as a whole (1-5). There are four main phases of wound healing: hemostasis, inflammation, proliferation, and remodeling, which direct the regeneration of damaged tissue. These phases overlap and require tightly regulated and coordinated signals (6,7), which often come in the form of chemokines (directing immune cells to the damaged tissue) and cytokines—which inform cells present in the milieu what purpose they should serve (8-10). Chronic wounds have been shown to have both dysregulated temporal control and absolute expression of these soluble chemokine and cytokine signals, leading to the stagnation of healing (10-12). There is a need for methods and compositions that modify the dysfunctional signaling environment of wounds to effect more efficient wound repair and treatment.

### SUMMARY OF INVENTION

The disclosure provides for methods for regenerating tissue, for preventing wound formation, or for treating wounds in a subject. The methods are achieved by administering a composition comprising a fms-related tyrosine kinase 3 ligand (Flt3L) polypeptide to the subject.

The composition may be administered intravenously or subcutaneously. Other forms of administration are described herein and may be implemented in the methods of the disclosure. The subject may include or exclude one that has cancer and/or has been diagnosed with cancer. The cancer may comprise head and neck cancer or a cancer described herein. The subject may include or exclude one that has been administered or has been prescribed radiation therapy and/or chemotherapy. The composition may be administered prior to the therapy, after the therapy, or concurrently with the therapy. The composition may be administered at a time period of within 24 hours of the therapy and either before or after the therapy. The composition may be administered within 24 hours before the administration of the radiation therapy. The composition may be administered within 24 hours after the administration of the radiation therapy. The composition may be administered, administered at least, or administered at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) hours before the radiation therapy. The composition may be administered, administered at least, or administered at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) hours after the radiation therapy.

The wound may may include or exclude skin atrophy, soft tissue fibrosis, desquamation, epithelial ulceration, fistula formation, and/or vessel rupture. The subject may include or exclude one that has diabetes. The subject may include or exclude one that has prediabetes. The diabetes may be type 2 diabetes. The diabetes may be type 1 diabetes. The method may comprise or further comprise treating the wound or a wound. The method may comprise treating radiation-induced oral mucositis (RIOM). The wound may include or exclude a chronic wound and/or the subject may include or exclude one that has chronic wounds. The subject may be one that has been determined to be at risk for chronic wounds. The method may provide for the increase in the frequence of dendritic cells within the tissue of the wound. The subject may have a non-healing wound or be at risk for having non-healing wounds. The wound may include or exclude an acute wound and/or the subject may include or exclude one that has acute wounds. The wound may include or exclude an infectious wound, ischemic wound, radiation poisoning wound, surgical wound, or ulcer. In the case where the wound is an ulcer, the ulcer may be further defined as an arterial ulcer, a venous ulcer, a diabetic ulcer, or a pressure ulcer. Methods of the disclosure may exclude the treatment of acute wounds. Methods of the disclosure may exclude the treatment of burn wounds. The subjects having acute or burn wounds may be excluded from the methods of the disclosure. The subject may include or exclude one that has undergone or will undergo wound debridement. The composition may be administered before, after, or concurrently with debridement of the wound. The composition may be administered within 24 hours of wound debridement. The composition may be administered, administered at least, or administered at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) hours before wound debridement. The composition may be administered, administered at least, or administered at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) hours after wound debridement. The composition may be administered as a single dose or in multiple doses, such as in at least two doses or at least 3 doses.

The subject may include or exclude one that has received or will receive (has been prescribed) an additional therapy. The subject may include or exclude one that has not received or has not been prescribed an additional therapy. The additional therapy may include or exclude an immunotherapy and/or an immune agonist.

The FLT3L polypeptide may include or exclude a fusion protein comprising: a FLT3L polypeptide linked to a serum protein. The serum protein may be albumin. The serum protein may be human serum albumin. The serum protein may be mouse serum albumin. The serum protein may comprise the amino acid sequence of SEQ ID NO:60, 61, or 64, or an amino acid sequence having at least 80% sequence identity to SEQ ID NO:60, 61, or 64. The serum protein may have an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:60, 61, or 64. The FLT3L polypeptide may comprise the amino acid sequence of one of SEQ ID NOs:40-50, 58 or 59 or an amino acid sequence having at least 80% sequence identity to one of SEQ ID NOs:40-50, 58 or 59. The FLT3L polypeptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs:40-50, 58 or 59. The FLT3L polypeptide may be further defined as an FLT3L extracellular domain. The FLT3L polypeptide may comprise a fusion protein comprising: a FLT3L extracellular domain operably linked to an immunoglobulin fragment crystallizable region (Fc region).

At least 5 amino acids may be truncated from the C-terminus of the FLT3L extracellular domain; and/or the Fc region does not comprise a hinge region. The FLT3L extracellular domain may be a human FLT3L extracellular domain or derived from a human FLT3L extracellular domain. The fusion protein may be capable of binding to human FLT3. The FLT3L extracellular domain may be from FLT3L isoform 1. The FLT3L extracellular domain may be from FLT3L isoform 2. The FLT3L extracellular domain may exclude the amino acid sequence PTAPQ. At least 6, 7, 8, 9, 10, 11, 12, 13, 14 or 15 amino acids (or any derivable range therein) are truncated from the C-terminus of the FLT3L extracellular domain. The FLT3L extracellular domain may exclude the amino acid sequence APTAPQ (SEQ ID NO:29), TAPTAPQ (SEQ ID NO:30), ATAPTAPQ (SEQ ID NO:31), EATAPTAPQ (SEQ ID NO:32), or LEATAPTAPQ (SEQ ID NO:33). The FLT3L extracellular domain may exclude the amino acid sequence PTAPQPP (SEQ ID NO:34), APTAPQPP (SEQ ID NO:35), TAPTAPQPP (SEQ ID NO:36), ATAPTAPQPP (SEQ ID NO:37), EATAPTAPQPP (SEQ ID NO:38), or LEATAPTAPQPP (SEQ ID NO:39). The FLT3L extracellular domain may comprise an N-terminal signal peptide. The FLT3L extracellular domain may comprise an amino acid substitution at one or more of the following amino acid positions: H8Y, K84E, N100, S102, N123 and S125, wherein the amino acid residue positions are with reference to SEQ ID NOs:1-18, 21-27 or 40-50. The FLT3L extracellular domain may comprise one or more of the following amino acid substitutions: H8Y, K84E, S102A, and/or S125A; wherein the amino acid residue positions are with reference to SEQ ID NOs:1-18, 21-27 or 40-50. One ore both of serine residues at positions 102 and 125 may be substituted to alanine, wherein the amino acid residue positions are with reference to SEQ ID NOs:1-18, 21-27 or 40-50.

The Fc region may include or exclude a Fc region from a human IgG1, IgG2, IgG3 or IgG4. The Fc region may comprise a human IgG1 isotype and comprises one or more amino acid substitutions in the Fc region at a residue position selected from the group consisting of: N297A, N297G, N297Q, N297G, D265A, L234A, L235A, C226S, C229S, P238S, E233P, L234V, P238A, A327Q, A327G, P329A, P329G, K322A, L234F, L235E, P331S, T394D, A330L, M252Y, S254T, T256E, M428L, N434S, T366W, T366S, L368A, Y407V, and any combination thereof, wherein the numbering of the residues is according to EU numbering. The Fc region may comprise a human IgG1 isotype and comprises one or more amino acid substitutions in the Fc region at a residue position selected from the group consisting of: L234A, L234V, L234F, L235A, L235E, P331S, and any combination thereof, wherein the numbering of the residues is according to EU numbering. The Fc region may comprise a human IgG4 isotype and comprises one or more amino acid substitutions in the Fc region at a residue position selected from the group consisting of: E233P, F234V, F234A, L235A, G237A, E318A, S228P, L235E, T394D, M252Y, S254T, T256E, N297A, N297G, N297Q, T366W, T366S, L368A, Y407V, M428L, N434S, and any combination thereof, wherein the numbering of the residues is according to EU numbering. The Fc region may comprise a human IgG4 isotype and comprises one or more amino acid substitutions in the Fc region at a residue position selected from the group consisting of: F234V, F234A, L235A, L235E, S228P, and any combination thereof, wherein the numbering of the residues is according to EU numbering. The Fc region may comprise the following amino acids at the indicated positions (EU index numbering): Tyrosine at position 252, threonine at position 254 and glutamic acid at position 256 (YTE); or Leucine at position 428 and serine at position 434 (LS). The FLT3L extracellular domain may comprise the amino acid sequence of one of SEQ ID NOs:40-50, or an amino acid sequence having at least 80% sequence identity to one of SEQ ID NOs:40-50. The FLT3L extracellular domain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any range derivable therein) sequence identity to one of SEQ ID NOs:40-50.

The Fc region may include or exclude the amino acid sequence of one of SEQ ID NOs:51-55, 62, and 63, or an amino acid sequence having at least 80% sequence identity to one of SEQ ID NOs:51-55, 62, and 63. The Fc region may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any range derivable therein) sequence identity to one of SEQ ID NOs:51-55, 62, and 63. The fusion protein may comprise the amino acid sequence of one of SEQ ID NOs:1-27, or an amino acid sequence having at least 80% sequence identity to one of SEQ ID NOs:1-27. The fusion protein may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any range derivable therein) sequence identity to one of SEQ ID NOs:1-27. The Fc region may be from a human IgG1 and does not comprise a hinge region. The C-terminus of the FLT3L extracellular domain may be un-truncated. The Fc region may be derived from a human IgG1 isotype and does not comprise a hinge region, e.g., does not the amino acid sequence EPKSCDKTHTCPPCP (SEQ ID NO:56) or EPKSCDKTHTCPPCPAPELL (SEQ ID NO:57). The Fc region may be from a human IgG4 and at least 5 amino acids are truncated from the C-terminus of the FLT3L extracellular domain. The Fc Region may comprise or further comprise a hinge region. The Fc region may be derived from a human IgG4 isotype and wherein at least 5 amino acids are truncated from the C-terminus of the FLT3L extracellular domain, e.g., wherein the FLT3L extracellular domain does not comprise the amino acid sequence PTAPQ.

The patient or subject may include or exclude one that has been previously treated for a condition or indication described herein. The patient or subject may include or exclude one that was resistant to the previous treatment. The patient or subject may include or exclude one that has been diagnosed with and/or is susceptible to a condition or indication described herein. The method may further comprise administration of an additional therapy, such as, for example, additional therapies described herein.

The terms “protein”, “polypeptide” and “peptide” are used interchangeably herein when referring to a gene product or synthetic amino acid polymer.

The terms “subject,” “mammal,” and “patient” are used interchangeably. The subject being treated may be a mammal. The subject may be a human. The subject may be a mouse, rat, rabbit, dog, donkey, sheep, goat, pig, or a laboratory test animal such as fruit fly, zebrafish, etc.

“Treatment” or treating may refer to any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. The treatment may exclude prevention of the disease.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Flt3L, both wild type (WT) and an engineered construct (MSA) improves wound closure in a mouse model of type 2 diabetic (db/db) mice. Left: WT Flt3L, at a dose of 10 μg, administered to the rear right hock immediately following wounding on day 0, improves wound closure in a mouse model of type 2 diabetes. Right: Long-lived, engineered Flt3L (MSA-Flt3L) reduces therapeutic dose of WT Flt3L 20-fold.

FIGS. 2A-2B: WT Flt3L demonstrates a trend towards increase of dendritic cells in diabetic wound tissue.

FIG. 3: Flt3L-SA protects against the effects of radiation induced ulceration of the tongue in a statistically significant manner. While Flt3L-Fc shows near significant trends in protection.

FIG. 4: Flt3L-SA increases DC cellularity in the tongue following irradiation. Decrease in efferocytic marker (Axl and Mertk) may be attributable to an increased efferocytic capacity of the DCs to clear wound debris in the irradiated tissue.

### DETAILED DESCRIPTION

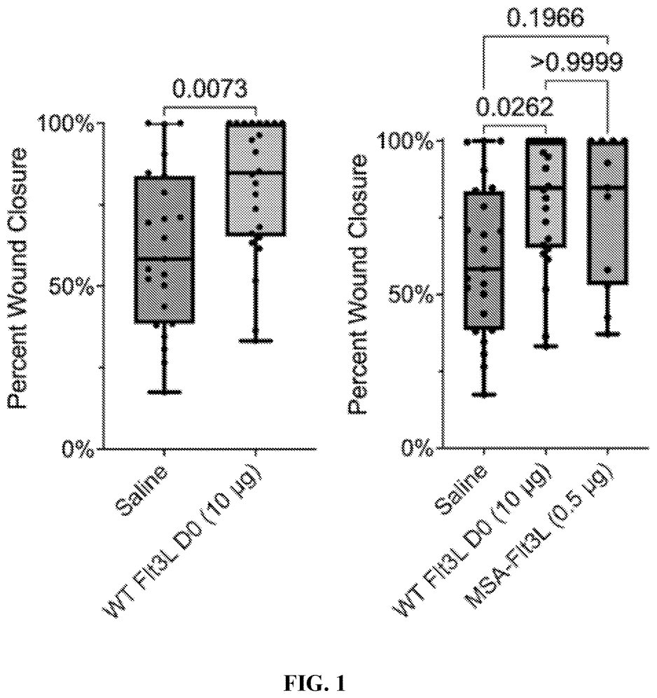

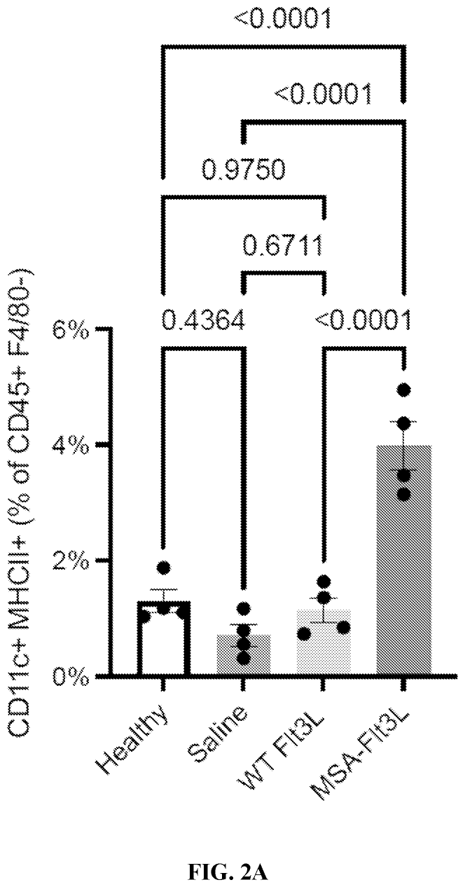

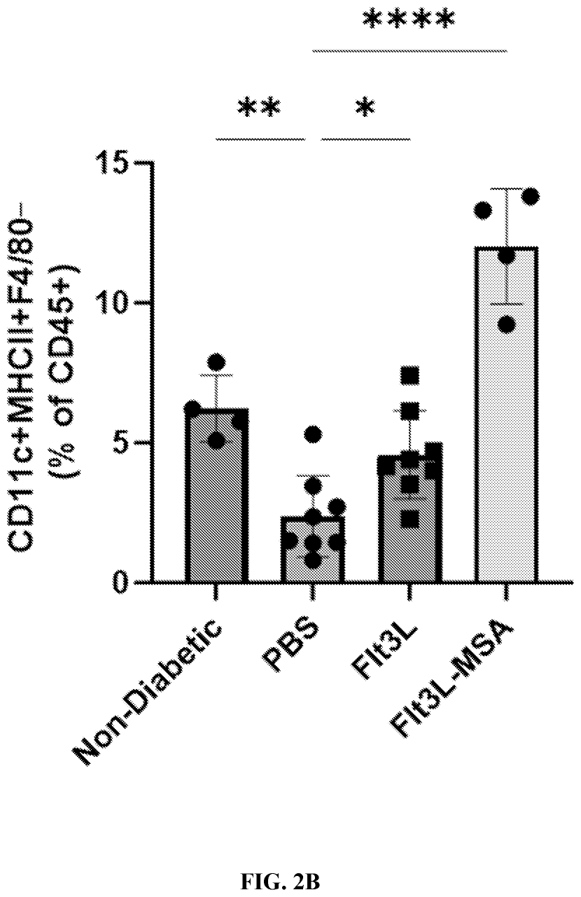

Flt3L is a soluble, focused signal that can expand the dendritic cell compartment. While it has been studied and utilized in many contexts, it has not been studied in the context of non-healing wounds. The inventors hypothesized that Flt3L can be used as a treatment for non-healing wounds. The examples of the application demonstrate that Flt3L heals chronic wounds in a type 2 diabetic mouse model (FIG. 1) and that Flt3L induces increase in DCs within the wound of type 2 diabetic mouse (FIG. 2).

### I. Flt3L Polypeptides

### A. Flt3L Proteins

Flt3L polypeptides may include those listed below:

| | | SEQ |
|---|---|---|
| Description | Sequence | ID NO: |
| murine | MTPDCYFSHSPISSNFKVKFRELTDHLLKDYPVTVAVNLQDEKHCKA | 58 |
| Flt3L | LWSLFLAQRWIEQLKTVAGSKMQTLLEDVNTEIHFVTSCTFQPLPECL | |
| | RFVQTNISHLLKDTCTQLLALKPCIGKACQNFSRCLEVQCQPDSSTLLP | |
| | PRSPIALEATELPEPRPRQ | |
| Human | MTQDCSFQHSPISSDFAVKIRELSDYLLQDYPVTVASNLQDEELCGGL | 59 |
| Flt3L | WRLVLAQRWMERLKTVAGSKMQGLLERVNTEIHFVTKCAFQPPPSC | |
| | LRFVQTNISRLLQETSEQLVALKPWITRQNFSRCLELQCQPDSSTLPPP | |
| | WSPRPLEATAPTA | |
| Flt3L | TQDCSFQHSPISSDFAVKIRELSDYLLQDYPVTVASNLQDEELCGGLW | 40 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRP | |
| Flt3L | TQDCSFQHSPISSDFAVKIRELSDYLLQDYPVTVASNLQDEELCGGLW | 41 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPL | |
| Flt3L | TQDCSFQHSPISSDFAVKIRELSDYLLQDYPVTVASNLQDEELCGGLW | 42 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLE | |
| Flt3L | TQDCSFQHSPISSDFAVKIRELSDYLLQDYPVTVASNLQDEELCGGLW | 43 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEA | |
| Flt3L | TQDCSFQHSPISSDFAVKIRELSDYLLQDYPVTVASNLQDEELCGGLW | 44 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEAT | |
| Flt3L | TQDCSFQHSPISSDFAVKIRELSDYLLQDYPVTVASNLQDEELCGGLW | 45 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATA | |
| Flt3L | TQDCSFQHSPISSDFAVKIRELSDYLLQDYPVTVASNLQDEELCGGLW | 46 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAP | |
| Flt3L | TQDCSFQHSPISSDFAVKIRELSDYLLQDYPVTVASNLQDEELCGGLW | 47 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPT | |
| Flt3L | TQDCSFQHSPISSDFAVKIRELSDYLLQDYPVTVASNLQDEELCGGLW | 48 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTA | |
| Flt3L | TQDCSFQHSPISSDFAVKIRELSDYLLQDYPVTVASNLQDEELCGGLW | 49 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTAP | |
| Flt3L | TQDCSFQHSPISSDFAVKIRELSDYLLQDYPVTVASNLQDEELCGGLW | 50 |
| polypeptide | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTAPQ | |

### B. Serum Proteins

The polypeptides of the disclosure may be further linked to a serum protein. Serum proteins include, for example, albumin, globulin, and fibrinogen. Globulins include alpha 1 globulins, alpha 2 globulins, beta globulins, and gamma globulins. The albumin may be mouse, human, bovine, or any other homologous albumin protein. The albumin may comprise human serum albumin, which is encoded by the ALB gene. The albumin may comprise mouse albumin.

| | | SEQ |
|---|---|---|
| Description | Sequence | ID NO: |
| Murine | EAHKSEIAHRYNDLGEQHFKGLVLIAFSQYLQKCSYDEHAKLVQEVT | 60 |
| Serum | DFAKTCVADESAANCDKSLHTLFGDKLCAIPNLRENYGELADCCTKQ | |
| Albumin | EPERNECFLQHKDDNPSLPPFERPEAEAMCTSFKENPTTFMGHYLHEV | |
| | ARRHPYFYAPELLYYAEQYNEILTQCCAEADKESCLTPKLDGVKEKA | |
| | LVSSVRQRMKCSSMQKFGERAFKAWAVARLSQTFPNADFAEITKLAT | |
| | DLTKVNKECCHGDLLECADDRAELAKYMCENQATISSKLQTCCDKP | |
| | LLKKAHCLSEVEHDTMPADLPAIAADFVEDQEVCKNYAEAKDVFLG | |
| | TFLYEYSRRHPDYSVSLLLRLAKKYEATLEKCCAEANPPACYGTVLA | |
| | EFQPLVEEPKNLVKTNCDLYEKLGEYGFQNAILVRYTQKAPQVSTPTL | |
| | VEAARNLGRVGTKCCTLPEDQRLPCVEDYLSAILNRVCLLHEKTPVSE | |
| | HVTKCCSGSLVERRPCFSALTVDETYVPKEFKAETFTFHSDICTLPEKE | |
| | KQIKKQTALAELVKHKPKATAEQLKTVMDDFAQFLDTCCKAADKDT | |
| | CFSTEGPNLVTRCKDALA | |
| Human | KWVTFISLLFLFSSAYSRGVFRRDAHKSEVAHRFKDLGEENFKALVLI | 61 |
| Serum | AFAQYLQQCPFEDHVKLVNEVTEFAKTCVADESAENCDKSLHTLFGD | |
| Albumin | KLCTVATLRETYGEMADCCAKQEPERNECFLQHKDDNPNLPRLVRPE | |
| | VDVMCTAFHDNEETFLKKYLYEIARRHPYFYAPELLFFAKRYKAAFT | |
| | ECCQAADKAACLLPKLDELRDEGKASSAKQRLKCASLQKFGERAFK | |
| | AWAVARLSQRFPKAEFAEVSKLVTDLTKVHTECCHGDLLECADDRA | |
| | DLAKYICENQDSISSKLKECCEKPLLEKSHCIAEVENDEMPADLPSLA | |
| | ADFVESKDVCKNYAEAKDVFLGMFLYEYARRHPDYSVVLLLRLAKT | |
| | YETTLEKCCAAADPHECYAKVFDEFKPLVEEPQNLIKQNCELFEQLGE | |
| | YKFQNALLVRYTKKVPQVSTPTLVEVSRNLGKVGSKCCKHPEAKRM | |
| | PCAEDYLSVVLNQLCVLHEKTPVSDRVTKCCTESLVNRRPCFSALEV | |
| | DETYVPKEFNAETFTFHADICTLSEKERQIKKQTALVELVKHKPKATK | |
| | EQLKAVMDDFAAFVEKCCKADDKETCFAEEGKKLVAASQAALGL | |

The serum protein may comprise a polypeptide of SEQ ID NO:42 or 43, or a fragment thereof, or a polypeptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to SEQ ID NO:42, 43, or a fragment thereof.

### C. Flt3L-Fc Fusion Proteins

Methods of the disclosure employ the use of FLT3L-Fc fusion proteins that have an extended serum half-life in a human subject, relative to soluble FLT3L.

The fusion protein may comprise a human fms related tyrosine kinase 3 ligand (FLT3L) extracellular domain operably linked to an immunoglobulin fragment crystallizable region (Fc region). The fusion protein may have least 5 amino acids are truncated from the C-terminus of the FLT3L extracellular domain. The Fc region may exclude a hinge region.

The FLT3L extracellular domain may be derived from a human FLT3L extracellular domain. The fusion protein may be capable of binding to human FLT3. The FLT3L extracellular domain may be from FLT3L isoform 1 or from FLT3L isoform 2. At least 6, 7, 8, 9, 10, 11, 12, 13, 14 or 15 amino acids may be truncated from the C-terminus of the FLT3L extracellular domain. The FLT3L extracellular domain may exclude (e.g., is deleted, removed or excluded) the amino acid sequence PTAPQ (SEQ ID NO:28), APTAPQ (SEQ ID NO:29), TAPTAPQ (SEQ ID NO:30), ATAPTAPQ (SEQ ID NO:31), EATAPTAPQ (SEQ ID NO:32), LEATAPTAPQ (SEQ ID NO:33), PTAPQPP (SEQ ID NO:34), APTAPQPP (SEQ ID NO:35), TAPTAPQPP (SEQ ID NO:36), ATAPTAPQPP (SEQ ID NO:37), EATAPTAPQPP (SEQ ID NO:38), or LEATAPTAPQPP (SEQ ID NO:39). The FLT3L extracellular domain may comprise or further comprise a N-terminal signal peptide. The FLT3L extracellular domain may comprise or further comprise one or more of the following amino acid substitutions: H8Y; K84E; S102A; and/or S125A; wherein the amino acid residue positions are with reference to SEQ ID NOs:1-18, 21-27 or 40-50. One or both of serine residues at positions 102 and 125 may be substituted to alanine, wherein the amino acid residue positions are with reference to SEQ ID NOs:1-18, 21-27 or 40-50.

The Fc region may be from a human IgG1, IgG2, IgG3 or IgG4. The Fc region may be from a human IgG1 or IgG4. The Fc region may comprise a human IgG1 isotype and include or exclude one or more amino acid substitutions in the Fc region at a residue position selected from the group consisting of: N297A, N297G, N297Q, N297G, D265A, L234A, L235A, C226S, C229S, P238S, E233P, L234V, P238A, A327Q, A327G, P329A, P329G, K322A, L234F, L235E, P331S, T394D, A330L, M252Y, S254T, T256E, M428L, N434S, T366W, T366S, L368A, Y407V, and any combination thereof, wherein the numbering of the residues is according to EU numbering. The Fc region may comprise a human IgG1 isotype and include or exclude one or more amino acid substitutions in the Fc region at a residue position selected from the group consisting of: L234A, L234V, L234F, L235A, L235E, P331S, and any combination thereof, wherein the numbering of the residues is according to EU numbering. The Fc region may comprise a human IgG4 isotype and include or exclude one or more amino acid substitutions in the Fc region at a residue position selected from the group consisting of: E233P, F234V, F234A, L235A, G237A, E318A, S228P, L235E, T394D, M252Y, S254T, T256E, N297A, N297G, N297Q, T366W, T366S, L368A, Y407V, M428L, N434S, and any combination thereof, wherein the numbering of the residues is according to EU numbering. The Fc region may comprise a human IgG4 isotype and include or exclude one or more amino acid substitutions in the Fc region at a residue position selected from the group consisting of: F234V, F234A, L235A, L235E, S228P, and any combination thereof, wherein the numbering of the residues is according to EU numbering. The Fc region may comprise the following amino acids at the indicated positions (EU index numbering): (i) Tyrosine at position 252, threonine at position 254 and glutamic acid at position 256 (YTE); or (ii) Leucine at position 428 and serine at position 434 (LS).

The FLT3L extracellular domain may comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to an amino acid sequence selected from the group consisting of SEQ ID NOs:40-50. The Fc region may comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to an amino acid sequence selected from the group consisting of SEQ ID NOs:51-55, 62, and 63. The fusion protein may comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to an amino acid sequence selected from the group consisting of SEQ ID NOs:1-18 and 21-27.

The Fc region may be from a human IgG1 and may exclude a hinge region. The C-terminus of the FLT3L extracellular domain may not be truncated. The fusion protein may comprise or consist of an amino acid sequence of SEQ ID NO:1. The fusion protein may comprise or consist of an amino acid sequence of SEQ ID NO:9. The fusion protein may comprise or consist of an amino acid sequence selected from the group consisting of SEQ ID NOs:1, 2, 5, 7, 9, 10, 13, 15, 22, 23 and 24, or comprise or consist of an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs:1, 2, 5, 7, 9, 10, 13, 15, 22, 23 and 24, wherein the Fc region is derived from a human IgG1 isotype and does not comprise a hinge region, e.g., does not the amino acid sequence EPKSCDKTHTCPPCP (SEQ ID NO:56) or EPKSCDKTHTCPPCPAPELL (SEQ ID NO:57). The Fc region may be from a human IgG4 and at least 5 amino acids may be truncated from the C-terminus of the FLT3L extracellular domain. The Fc region may comprise a hinge region. The fusion protein may comprise or consist of an amino acid sequence of SEQ ID NO:6. The fusion protein may comprise or consist of an amino acid sequence of SEQ ID NO:14. The fusion protein may comprise or consist of an amino acid sequence selected from the group consisting of SEQ ID NOs:3, 4, 6, 8, 11, 12, 14, 16, 17, 18, 25 and 26, or comprise or consist of an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs:3, 4, 6, 8, 11, 12, 14, 16, 17, 18, 25 and 26, wherein the Fc region is derived from a human IgG4 isotype and wherein at least 5 amino acids are truncated from the C-terminus of the FLT3L extracellular domain, e.g., wherein the FLT3L extracellular domain does not comprise the amino acid sequence PTAPQ (SEQ ID NO:28).

The fusion protein may comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to an amino acid sequence selected from the group consisting of SEQ ID NOs:19-20.

Fc regions, fusion proteins, and Flt3L polypeptides useful in the disclosure are described below:

| | | SEQ |
|---|---|---|
| Description | Sequence | ID NO: |
| Murine | AKTTPPSVYPLAPGSAAQTNSMVTLGCLVKGYFPEPVTVTWNSGSLS | 62 |
| IgG1 Fc | SGVHTFPAVLQSDLYTLSSSVTVPSSPRPSETVTCNVAHPASSTKVDK | |
| | KIVPRDCGCKPCICTVPEVSSVFIFPPKPKDVLTITLTPKVTCVVVDISK | |
| | DDPEVQFSWFVDDVEVHTAQTQPREEQFNSTFRSVSELPIMHQDWLN | |
| | GKEFKCRVNSAAFPAPIEKTISKTKGRPKAPQVYTIPPPKEQMAKDKV | |
| | SLTCMITDFFPEDITVEWQWNGQPAENYKNTQPIMNTNGSYFVYSKL | |
| | NVQKSNWEAGNTFTCSVLHEGLHNHHTEKSLSHSPGK | |
| Human | ASTKGPSVFPLAPSSKSTSGGTAALGCLVKDYFPEPVTVSWNSGALTS | 63 |
| IgG1 Fc | GVHTFPAVLQSSGLYSLSSVVTVPSSSLGTQTYICNVNHKPSNTKVDK | |
| | KVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSR | |
| | DELTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| Fusion | TQDCSFQHSPISSDFAVKIRELSDYLLQDYPVTVASNLQDEELCGGLW | 1 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTAPQGGPSVFLFPPKPKDTLMISRTPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTLPPSREEMTKNQVS | |
| | LTCLVKGFYPSDIAVEWESNGQPENNYKTTPPVLDSDGSFFLYSKLTV | |
| | DKSRWQQGNVFSCSVMHEALHNHYTQKSLSLSPGK | |
| Fusion | TQDCSFQHSPISSDFAVKIRELSDYLLQDYPVTVASNLQDEELCGGLW | 2 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAGGPSVFLFPPKPKDTLMISRTPEVTCVVVDVSHEDPEVKF | |
| | NWYVDGVEVHNAKTKPREEQYNSTYRVVSVLTVLHQDWLNGKEYK | |
| | CKVSNKALPAPIEKTISKAKGQPREPQVYTLPPSREEMTKNQVSLTCL | |
| | VKGFYPSDIAVEWESNGQPENNYKTTPPVLDSDGSFFLYSKLTVDKSR | |
| | WQQGNVFSCSVMHEALHNHYTQKSLSLSPGK | |
| Fusion | TQDCSFQHSPISSDFAVKIRELSDYLLQDYPVTVASNLQDEELCGGLW | 3 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTAPQESKYGPPCPPCPAPEFEGGPSVFLFPPKPKDTLMISR | |
| | TPEVTCVVVDVSQEDPEVQFNWYVDGVEVHNAKTKPREEQFNSTYR | |
| | VVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPP | |
| | VLDSDGSFFLYSRLTVDKSRWQEGNVFSCSVMHEALHNHYTQKSLSL | |
| | SLGK | |
| Fusion | TQDCSFQHSPISSDFAVKIRELSDYLLQDYPVTVASNLQDEELCGGLW | 4 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTAPQESKYGPPCPPCPAPEAAGGPSVFLFPPKPKDTLMIS | |
| | RTPEVTCVVVDVSQEDPEVQFNWYVDGVEVHNAKTKPREEQFNSTY | |
| | RVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTT | |
| | PPVLDSDGSFFLYSRLTVDKSRWQEGNVFSCSVMHEALHNHYTQKSL | |
| | SLSLGK | |
| Fusion | TQDCSFQHSPISSDFAVKIRELSDYLLQDYPVTVASNLQDEELCGGLW | 5 |
| Protein | RLVLAQRWMERLKTVAGSKMQGLLERVNTEIHFVTKCAFQPPPSCLR | |
| | FVQTNIARLLQETSEQLVALKPWITRQNFARCLELQCQPDSSTLPPPW | |
| | SPRPLEATAPTAPQGGPSVFLFPPKPKDTLMISRTPEVTCVVVDVSHED | |
| | PEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQV | |
| | SLTCLVKGFYPSDIAVEWESNGQPENNYKTTPPVLDSDGSFFLYSKLT | |
| | VDKSRWQQGNVFSCSVMHEALHNHYTQKSLSLSPGK | |
| Fusion | TQDCSFQHSPISSDFAVKIRELSDYLLQDYPVTVASNLQDEELCGGLW | 6 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAESKYGPPCPPCPAPEAAGGPSVFLFPPKPKDTLMISRTPEV | |
| | TCVVVDVSQEDPEVQFNWYVDGVEVHNAKTKPREEQFNSTYRVVSV | |
| | LTVLHQDWLNGKEYKCKVSNKGLPSSIEKTISKAKGQPREPQVYTLPP | |
| | SQEEMTKNQVSLTCLVKGFYPSDIAVEWESNGQPENNYKTTPPVLDS | |
| | DGSFFLYSRLTVDKSRWQEGNVFSCSVMHEALHNHYTQKSLSLSLGK | |
| Fusion | TQDCSFQHSPISSDFAVKIRELSDYLLQDYPVTVASNLQDEELCGGLW | 7 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSN | |
| | KALPAPIEKTISKAKGQPREPQVYTLPPSREEMTKNQVSLTCLVKGFY | |
| | PSDIAVEWESNGQPENNYKTTPPVLDSDGSFFLYSKLTVDKSRWQQG | |
| | NVFSCSVMHEALHNHYTQKSLSLSPGK | |
| Fusion | TQDCSFQHSPISSDFAVKIRELSDYLLQDYPVTVASNLQDEELCGGLW | 8 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPESKYGPPCPPCPAPEAAGGPSVFLFPPKPKDTLMISRTPEVTCVVV | |
| | DVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQEE | |
| | MTKNQVSLTCLVKGFYPSDIAVEWESNGQPENNYKTTPPVLDSDGSF | |
| | FLYSRLTVDKSRWQEGNVFSCSVMHEALHNHYTQKSLSLSLGK | |
| Fusion | TQDCSFQHSPISSDFAVKIRELSDYLLQDYPVTVASNLQDEELCGGLW | 9 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTAPQGGPSVFLFPPKPKDTLYITREPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTLPPSREEMTKNQVS | |
| | LTCLVKGFYPSDIAVEWESNGQPENNYKTTPPVLDSDGSFFLYSKLTV | |
| | DKSRWQQGNVFSCSVMHEALHNHYTQKSLSLSPGK | |
| Fusion | TQDCSFQHSPISSDFAVKIRELSDYLLQDYPVTVASNLQDEELCGGLW | 10 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAGGPSVFLFPPKPKDTLYITREPEVTCVVVDVSHEDPEVKF | |
| | NWYVDGVEVHNAKTKPREEQYNSTYRVVSVLTVLHQDWLNGKEYK | |
| | CKVSNKALPAPIEKTISKAKGQPREPQVYTLPPSREEMTKNQVSLTCL | |
| | VKGFYPSDIAVEWESNGQPENNYKTTPPVLDSDGSFFLYSKLTVDKSR | |
| | WQQGNVFSCSVMHEALHNHYTQKSLSLSPGK | |
| Fusion | TQDCSFQHSPISSDFAVKIRELSDYLLQDYPVTVASNLQDEELCGGLW | 11 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTAPQESKYGPPCPPCPAPEFEGGPSVFLFPPKPKDTLYITR | |
| | EPEVTCVVVDVSQEDPEVQFNWYVDGVEVHNAKTKPREEQFNSTYR | |
| | VVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPP | |
| | VLDSDGSFFLYSRLTVDKSRWQEGNVFSCSVMHEALHNHYTQKSLSL | |
| | SLGK | |
| Fusion | TQDCSFQHSPISSDFAVKIRELSDYLLQDYPVTVASNLQDEELCGGLW | 12 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTAPQESKYGPPCPPCPAPEAAGGPSVFLFPPKPKDTLYIT | |
| | REPEVTCVVVDVSQEDPEVQFNWYVDGVEVHNAKTKPREEQFNSTY | |
| | RVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTT | |
| | PPVLDSDGSFFLYSRLTVDKSRWQEGNVFSCSVMHEALHNHYTQKSL | |
| | SLSLGK | |
| Fusion | TQDCSFQHSPISSDFAVKIRELSDYLLQDYPVTVASNLQDEELCGGLW | 13 |
| Protein | RLVLAQRWMERLKTVAGSKMQGLLERVNTEIHFVTKCAFQPPPSCLR | |
| | FVQTNIARLLQETSEQLVALKPWITRQNFARCLELQCQPDSSTLPPPW | |
| | SPRPLEATAPTAPQGGPSVFLFPPKPKDTLYITREPEVTCVVVDVSHED | |
| | PEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQV | |
| | SLTCLVKGFYPSDIAVEWESNGQPENNYKTTPPVLDSDGSFFLYSKLT | |
| | VDKSRWQQGNVFSCSVMHEALHNHYTQKSLSLSPGK | |
| Fusion | TQDCSFQHSPISSDFAVKIRELSDYLLQDYPVTVASNLQDEELCGGLW | 14 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAESKYGPPCPPCPAPEAAGGPSVFLFPPKPKDTLYITREPEV | |
| | TCVVVDVSQEDPEVQFNWYVDGVEVHNAKTKPREEQFNSTYRVVSV | |
| | LTVLHQDWLNGKEYKCKVSNKGLPSSIEKTISKAKGQPREPQVYTLPP | |
| | SQEEMTKNQVSLTCLVKGFYPSDIAVEWESNGQPENNYKTTPPVLDS | |
| | DGSFFLYSRLTVDKSRWQEGNVFSCSVMHEALHNHYTQKSLSLSLGK | |
| Fusion | TQDCSFQHSPISSDFAVKIRELSDYLLQDYPVTVASNLQDEELCGGLW | 15 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPGGPSVFLFPPKPKDTLYITREPEVTCVVVDVSHEDPEVKFNWYVD | |
| | GVEVHNAKTKPREEQYNSTYRVVSVLTVLHQDWLNGKEYKCKVSN | |
| | KALPAPIEKTISKAKGQPREPQVYTLPPSREEMTKNQVSLTCLVKGFY | |
| | PSDIAVEWESNGQPENNYKTTPPVLDSDGSFFLYSKLTVDKSRWQQG | |
| | NVFSCSVMHEALHNHYTQKSLSLSPGK | |
| Fusion | TQDCSFQHSPISSDFAVKIRELSDYLLQDYPVTVASNLQDEELCGGLW | 16 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPESKYGPPCPPCPAPEAAGGPSVFLFPPKPKDTLYITREPEVTCVVV | |
| | DVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQEE | |
| | MTKNQVSLTCLVKGFYPSDIAVEWESNGQPENNYKTTPPVLDSDGSF | |
| | FLYSRLTVDKSRWQEGNVFSCSVMHEALHNHYTQKSLSLSLGK | |
| Fusion | TQDCSFQHSPISSDFAVKIRELSDYLLQDYPVTVASNLQDEELCGGLW | 17 |
| Protein | RLVLAQRWMERLKTVAGSKMQGLLERVNTEIHFVTKCAFQPPPSCLR | |
| | FVQTNIARLLQETSEQLVALKPWITRQNFARCLELQCQPDSSTLPPPW | |
| | SPRPESKYGPPCPPCPAPEAAGGPSVFLFPPKPKDTLMISRTPEVTCVV | |
| | VDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEE | |
| | MTKNQVSLTCLVKGFYPSDIAVEWESNGQPENNYKTTPPVLDSDGSF | |
| | FLYSRLTVDKSRWQEGNVFSCSVMHEALHNHYTQKSLSLSLGK | |
| Fusion | TQDCSFQHSPISSDFAVKIRELSDYLLQDYPVTVASNLQDEELCGGLW | 18 |
| Protein | RLVLAQRWMERLKTVAGSKMQGLLERVNTEIHFVTKCAFQPPPSCLR | |
| | FVQTNIARLLQETSEQLVALKPWITRQNFARCLELQCQPDSSTLPPPW | |
| | SPRPESKYGPPCPPCPAPEAAGGPSVFLFPPKPKDTLYITREPEVTCVV | |
| | VDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEE | |
| | MTKNQVSLTCLVKGFYPSDIAVEWESNGQPENNYKTTPPVLDSDGSF | |
| | FLYSRLTVDKSRWQEGNVFSCSVMHEALHNHYTQKSLSLSLGK | |
| Fusion | TPDCYFSHSPISSNFKVKFRELTDHLLKDYPVTVAVNLQDEKHCKAL | 19 |
| Protein | WSLFLAQRWIEQLKTVAGSKMQTLLEDVNTEIHFVTSCTFQPLPECLR | |
| | FVQTNISHLLKDTCTQLLALKPCIGKACQNFSRCLEVQCQPDSSTLLPP | |
| | RSPIALEATELPEPRGPTIKPCPPCKCPAPNAAGGPSVFIFPPKIKDVLMI | |
| | SLSPIVTCVVVDVSEDDPDVQISWFVNNVEVHTAQTQTHREDYNSTL | |
| | RVVSALPIQHQDWMSGKEFKCKVNNKDLGAPIERTISKPKGSVRAPQ | |
| | VYVLPPPEEEMTKKQVTLTCMVTDFMPEDIYVEWTNNGKTELNYKN | |
| | TEPVLDSDGSYFMYSKLRVEKKNWVERNSYSCSVVHEGLHNHHTTK | |
| | SFSRTPGK | |
| Fusion | TPDCYFSHSPISSNFKVKFRELTDHLLKDYPVTVAVNLQDEKHCKAL | 20 |
| Protein | WSLFLAQRWIEQLKTVAGSKMQTLLEDVNTEIHFVTSCTFQPLPECLR | |
| | FVQTNISHLLKDTSTQLLALKPCIGKACQNFSRCLEVQCQPDSSTLLPP | |
| | RSPIALEATELPEPRGPTIKPCPPCKCPAPNAAGGPSVFIFPPKIKDVLMI | |
| | SLSPIVTCVVVDVSEDDPDVQISWFVNNVEVHTAQTQTHREDYNSTL | |
| | RVVSALPIQHQDWMSGKEFKCKVNNKDLGAPIERTISKPKGSVRAPQ | |
| | VYVLPPPEEEMTKKQVTLTCMVTDFMPEDIYVEWTNNGKTELNYKN | |
| | TEPVLDSDGSYFMYSKLRVEKKNWVERNSYSCSVVHEGLHNHHTTK | |
| | SFSRTPGK | |
| Fusion | TQDCSFQHSPISSDFAVKIRELSDYLLQDYPVTVASNLQDEELCGGLW | 21 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTAPQGGPSVFLFPPKPKDTLMISRTPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTKPPSREEMTKNQVS | |
| | LSCLVKGFYPSDIAVEWESNGQPENNYKTTVPVLDSDGSFRLASYLTV | |
| | DKSRWQQGNVFSCSVMHEALHNHYTQKSLSLSPGK | |
| Fusion | TQDCSFQYSPISSDFAVKIRELSDYLLQDYPVTVASNLQDEELCGGLW | 22 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDSSTLPPPWS | |
| | PRPLEATAPTAPQGGPSVFLFPPKPKDTLMISRTPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTLPPSREEMTKNQVS | |
| | LTCLVKGFYPSDIAVEWESNGQPENNYKTTPPVLDSDGSFFLYSKLTV | |
| | DKSRWQQGNVFSCSVMHEALHNHYTQKSLSLSPGK | |
| Fusion | TQDCSFQHSPISSDFAVKIRELSDYLLQDYPVTVASNLQDEELCGGLW | 23 |
| Protein | RLVLAQRWMERLKTVAGSKMQGLLERVNTEIHFVTECAFQPPPSCLR | |
| | FVQTNISRLLQETSEQLVALKPWITRQNFSRCLELQCQPDSSTLPPPWS | |
| | PRPLEATAPTAPQGGPSVFLFPPKPKDTLMISRTPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTLPPSREEMTKNQVS | |
| | LTCLVKGFYPSDIAVEWESNGQPENNYKTTPPVLDSDGSFFLYSKLTV | |
| | DKSRWQQGNVFSCSVMHEALHNHYTQKSLSLSPGK | |
| Fusion | TQDCSFQYSPISSDFAVKIRELSDYLLQDYPVTVASNLQDEELCGGLW | 24 |
| Protein | RLVLAQRWMERLKTVAGSKMQGLLERVNTEIHFVTECAFQPPPSCLR | |
| | FVQTNISRLLQETSEQLVALKPWITRQNFSRCLELQCQPDSSTLPPPWS | |
| | PRPLEATAPTAPQGGPSVFLFPPKPKDTLMISRTPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTLPPSREEMTKNQVS | |
| | LTCLVKGFYPSDIAVEWESNGQPENNYKTTPPVLDSDGSFFLYSKLTV | |
| | DKSRWQQGNVFSCSVMHEALHNHYTQKSLSLSPGK | |
| Fusion | TQDCSFQHSPISSDFAVKIRELSDYLLQDYPVTVASNLQDEELCGGLW | 25 |
| Protein | RLVLAQRWMERLKTVAGSKMQGLLERVNTEIHFVTKCAFQPPPSCLR | |
| | FVQTNIARLLQETSEQLVALKPWITRQNFARCLELQCQPDSSTLPPPW | |
| | SPRPLEATAESKYGPPCPPCPAPEAAGGPSVFLFPPKPKDTLYITREPEV | |
| | TCVVVDVSQEDPEVQFNWYVDGVEVHNAKTKPREEQFNSTYRVVSV | |
| | LTVLHQDWLNGKEYKCKVSNKGLPSSIEKTISKAKGQPREPQVYTLPP | |
| | SQEEMTKNQVSLTCLVKGFYPSDIAVEWESNGQPENNYKTTPPVLDS | |
| | DGSFFLYSRLTVDKSRWQEGNVFSCSVMHEALHNHYTQKSLSLSLGK | |
| Fusion | TQDCSFQHSPISSDFAVKIRELSDYLLQDYPVTVASNLQDEELCGGLW | 26 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDAAALPPPW | |
| | SPRPLEATAESKYGPPCPPCPAPEAAGGPSVFLFPPKPKDTLYITREPEV | |
| | TCVVVDVSQEDPEVQFNWYVDGVEVHNAKTKPREEQFNSTYRVVSV | |
| | LTVLHQDWLNGKEYKCKVSNKGLPSSIEKTISKAKGQPREPQVYTLPP | |
| | SQEEMTKNQVSLTCLVKGFYPSDIAVEWESNGQPENNYKTTPPVLDS | |
| | DGSFFLYSRLTVDKSRWQEGNVFSCSVMHEALHNHYTQKSLSLSLGK | |
| Fusion | TQDCSFQHSPISSDFAVKIRELSDYLLQDYPVTVASNLQDEELCGGLW | 27 |
| Protein | RLVLAQRWMERLKTVAGSKMQGLLERVNTEIHFVTKCAFQPPPSCLR | |
| | FVQTNISRLLQETSEQLVALKPWITRQNFSRCLELQCQPDAAALPPPW | |
| | APRPLEATAEAKYGPPCPPCPAPEAAGGPSVFLFPPKPKDTLYITREPE | |
| | VTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTL | |
| | PPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSL | |
| | GK | |
| Fc region | GGPSVFLFPPKPKDTLMISRTPEVTCVVVDVSHEDPEVKFNWYVDGV | 51 |
| | EVHNAKTKPREEQYNSTYRVVSVLTVLHQDWLNGKEYKCKVSNKA | |
| | LPAPIEKTISKAKGQPREPQVYTLPPSREEMTKNQVSLTCLVKGFYPSD | |
| | IAVEWESNGQPENNYKTTPPVLDSDGSFFLYSKLTVDKSRWQQGNVF | |
| | SCSVMHEALHNHYTQKSLSLSPGK | |
| Fc region | GGPSVFLFPPKPKDTLYITREPEVTCVVVDVSHEDPEVKFNWYVDGV | 52 |
| | EVHNAKTKPREEQYNSTYRVVSVLTVLHQDWLNGKEYKCKVSNKA | |
| | LPAPIEKTISKAKGQPREPQVYTLPPSREEMTKNQVSLTCLVKGFYPSD | |
| | IAVEWESNGQPENNYKTTPPVLDSDGSFFLYSKLTVDKSRWQQGNVF | |
| | SCSVMHEALHNHYTQKSLSLSPGK | |
| Fc region | ESKYGPPCPPCPAPEFEGGPSVFLFPPKPKDTLMISRTPEVTCVVVDVS | 53 |
| | QEDPEVQFNWYVDGVEVHNAKTKPREEQFNSTYRVVSVLTVLHQD | |
| | WLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYS | |
| | RLTVDKSRWQEGNVFSCSVMHEALHNHYTQKSLSLSLGK | |
| Fc region | ESKYGPPCPPCPAPEAAGGPSVFLFPPKPKDTLMISRTPEVTCVVVDVS | 54 |
| | QEDPEVQFNWYVDGVEVHNAKTKPREEQFNSTYRVVSVLTVLHQD | |
| | WLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYS | |
| | RLTVDKSRWQEGNVFSCSVMHEALHNHYTQKSLSLSLGK | |
| Fc region | ESKYGPPCPPCPAPEFEGGPSVFLFPPKPKDTLYITREPEVTCVVVDVS | 55 |
| | QEDPEVQFNWYVDGVEVHNAKTKPREEQFNSTYRVVSVLTVLHQD | |
| | WLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYS | |
| | RLTVDKSRWQEGNVFSCSVMHEALHNHYTQKSLSLSLGK | |

### II. Polypeptides

As used herein, a “protein” or “polypeptide” refers to a molecule comprising at least five amino acid residues. As used herein, the term “wild-type” refers to the endogenous version of a molecule that occurs naturally in an organism. Wild-type versions of a protein or polypeptide may be employed or a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. A modified/variant protein or polypeptide may have at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. The disclosure also provides for isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs:1-64.

The protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs:1-64.

The protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:1-64.

The polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs:1-64 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOs:1-64.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOs:1-64 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOs:1-64.

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 (or any derivable range therein) of any of SEQ ID NOs:1-64 and may be a substitution with any amino acid or may be a substitution with an alanine, arginine, asparagine, aspartic acid, cysteine, glutamine, glutamic acid, glycine, histidine, isoleucine, leusine, lysine, methionine, phenylilacnine, proline, serine, threonine, tryptophan, tyrosine, or valine.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about
0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylilacnine to tyrosine, leucine or methionine; serine to threonine; threonine to serine; tryptophan to tyrosine; tyrosine to tryptophan or phenylilacnine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### B. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. Areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylilacnine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, the substitution of amino acids whose hydropathy indices are within ±2 is included. In some aspects of the present disclosure, those that are within ±1 are included, and in other aspects of the present disclosure, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. The greatest local average hydrophilicity of a protein, as governed by the hydrophilicity of its adjacent amino acids, may correlate with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylilacnine (−2.5); and tryptophan (−3.4). In making changes based upon similar hydrophilicity values, the substitution of amino acids whose hydrophilicity values are within ±2 are included, in other embodiments, those which are within ±1 are included, and those within ±0.5 may also be included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

Amino acid substitutions that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides may be made. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### III. Nucleic Acids

The current disclosure also describes recombinant polynucleotides encoding the proteins, polypeptides, and peptides of the disclosure.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including all values and ranges there between, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

The disclosure also describes isolated nucleic acid segments and recombinant vectors incorporating nucleic acid sequences that encode a polypeptide or peptide of the disclosure. The term “recombinant” may be used in conjunction with a polynucleotide or polypeptide and generally refers to a polypeptide or polynucleotide produced and/or manipulated in vitro or that is a replication product of such a molecule.

The disclosure also describes isolated nucleic acid segments and recombinant vectors incorporating nucleic acid sequences that encode a polypeptide or peptide of the disclosure.

The nucleic acid segments used in the current disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

The disclosure also describes polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

The disclosure also contemplates the use of polynucleotides which are complementary to all the above described polynucleotides.

### A. Vectors

Polypeptides of the disclosure may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNA, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). In addition to encoding a polypeptide of the disclosure, the vector can encode other polypeptide sequences such as a one or more other bacterial peptide, a tag, or an immunogenicity enhancing peptide. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. The vector may comprise pSeqTag-A or pcDNA3.1.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### B. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and may direct high level expression of the introduced DNA segment under specified conditions, such as large-scale production of recombinant proteins or peptides.

Various elements/promoters may be employed in the context of the present invention to regulate the expression of a gene. Examples of such inducible elements, which are regions of a nucleic acid sequence that can be activated in response to a specific stimulus, include but are not limited to Immunoglobulin Heavy Chain (Banerji et al., 1983; Gilles et al., 1983; Grosschedl et al., 1985; Atchinson et al., 1986, 1987; Imler et al., 1987; Weinberger et al., 1984; Kiledjian et al., 1988; Porton et al.; 1990), Immunoglobulin Light Chain (Queen et al., 1983; Picard et al., 1984), T Cell Receptor (Luria et al., 1987; Winoto et al., 1989; Redondo et al.; 1990), HLA DQ α and/or DQ β Sullivan et al., 1987), γ Interferon (Goodbourn et al., 1986; Fujita et al., 1987; Goodbourn et al., 1988), Interleukin-2 (Greene et al., 1989), Interleukin-2 Receptor (Greene et al., 1989; Lin et al., 1990), MHC Class II 5 (Koch et al., 1989), MHC Class II HLA-DR Sherman et al., 1989), β-Actin (Kawamoto et al., 1988; Ng et al.; 1989), Muscle Creatine Kinase (MCK) (Jaynes et al., 1988; Horlick et al., 1989; Johnson et al., 1989), Prealbumin (Transthyretin) (Costa et al., 1988), Elastase I (Ornitz et al., 1987), Metallothionein (MTII) (Karin et al., 1987; Culotta et al., 1989), Collagenase (Pinkert et al., 1987; Angel et al., 1987), Albumin (Pinkert et al., 1987; Tronche et al., 1989, 1990), □-Fetoprotein (Godbout et al., 1988; Campere et al., 1989), γ-Globin (Bodine et al., 1987; Perez-Stable et al., 1990), β-Globin (Trudel et al., 1987), c-fos (Cohen et al., 1987), c-Ha-Ras (Triesman, 1986; Deschamps et al., 1985), Insulin (Edlund et al., 1985), Neural Cell Adhesion Molecule (NCAM) (Hirsh et al., 1990), α1-Antitrypain (Latimer et al., 1990), H2B (TH2B) Histone (Hwang et al., 1990), Mouse and/or Type I Collagen (Ripe et al., 1989), Glucose-Regulated Proteins (GRP94 and GRP78) (Chang et al., 1989), Rat Growth Hormone (Larsen et al., 1986), Human Serum Amyloid A (SAA) (Edbrooke et al., 1989), Troponin I (TN I) (Yutzey et al., 1989), Platelet-Derived Growth Factor (PDGF) (Pech et al., 1989), Duchenne Muscular Dystrophy (Klamut et al., 1990), SV40 (Banerji et al., 1981; Moreau et al., 1981; Sleigh et al., 1985; Firak et al., 1986; Herr et al., 1986; Imbra et al., 1986; Kadesch et al., 1986; Wang et al., 1986; Ondek et al., 1987; Kuhl et al., 1987; Schaffner et al., 1988), Polyoma (Swartzendruber et al., 1975; Vasseur et al., 1980; Katinka et al., 1980, 1981; Tyndell et al., 1981; Dandolo et al., 1983; de Villiers et al., 1984; Hen et al., 1986; Satake et al., 1988; Campbell et al., 1988), Retroviruses (Kriegler et al., 1982, 1983; Levinson et al., 1982; Kriegler et al., 1983, 1984a, b, 1988; Bosze et al., 1986; Miksicek et al., 1986; Celander et al., 1987; Thiesen et al., 1988; Celander et al., 1988; Choi et al., 1988; Reisman et al., 1989), Papilloma Virus (Campo et al., 1983; Lusky et al., 1983; Spandidos and Wilkie, 1983; Spalholz et al., 1985; Lusky et al., 1986; Cripe et al., 1987; Gloss et al., 1987; Hirochika et al., 1987; Stephens et al., 1987), Hepatitis B Virus (Bulla et al., 1986; Jameel et al., 1986; Shaul et al., 1987; Spandau et al., 1988; Vannice et al., 1988), Human Immunodeficiency Virus (Muesing et al., 1987; Hauber et al., 1988; Jakobovits et al., 1988; Feng et al., 1988; Takebe et al., 1988; Rosen et al., 1988; Berkhout et al., 1989; Laspia et al., 1989; Sharp et al., 1989; Braddock et al., 1989), Cytomegalovirus (CMV) IE (Weber et al., 1984; Boshart et al., 1985; Foecking et al., 1986), Gibbon Ape Leukemia Virus (Holbrook et al., 1987; Quinn et al., 1989).

Inducible elements include, but are not limited to MT II-Phorbol Ester (TFA)/Heavy metals (Palmiter et al., 1982; Haslinger et al., 1985; Searle et al., 1985; Stuart et al., 1985; Imagawa et al., 1987, Karin et al., 1987; Angel et al., 1987b; McNeall et al., 1989); MMTV (mouse mammary tumor virus)-Glucocorticoids (Huang et al., 1981; Lee et al., 1981; Majors et al., 1983; Chandler et al., 1983; Lee et al., 1984; Ponta et al., 1985; Sakai et al., 1988); γ-Interferon-poly(rI)x/poly(rc) (Tavernier et al., 1983); Adenovirus 5 E2-EIA (Imperiale et al., 1984); Collagenase-Phorbol Ester (TPA) (Angel et al., 1987a); Stromelysin-Phorbol Ester (TPA) (Angel et al., 1987b); SV40-Phorbol Ester (TPA) (Angel et al., 1987b); Murine MX Gene-Interferon, Newcastle Disease Virus (Hug et al., 1988); GRP78 Gene-A23187 (Resendez et al., 1988); β-2-Macroglobulin-IL-6 (Kunz et al., 1989); Vimentin-Serum (Rittling et al., 1989); MHC Class I Gene H-2Kb-Interferon (Blanar et al., 1989); HSP70-EIA/SV40 Large T Antigen (Taylor et al., 1989, 1990a, 1990b); Proliferin-Phorbol Ester/TPA (Mordacq et al., 1989); Tumor Necrosis Factor-PMA (Hensel et al., 1989); and Thyroid Stimulating Hormone Alpha Gene-Thyroid Hormone (Chatterjee et al., 1989).

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

### C. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

The use of internal ribosome entry sites (IRES) elements may be used to create multigene, or polycistronic, messages. IRES elements are able to bypass the ribosome scanning model of 5′ methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

### D. Selectable and Screenable Markers

In certain embodiments of the invention, cells containing a nucleic acid construct of the current disclosure may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. As an alternative, 2A peptides could be used to introduce ribosomal skips to enable expression of multiple polypeptidic or protein sequences.

### E. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

### F. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL□ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### IV. Compositions

In some embodiments, pharmaceutical compositions are administered to a subject. Different aspects involve administering an effective amount of a composition to a subject. In some embodiments, a composition comprising a peptide of the disclosure may be administered to the subject or patient to treat wounds or facilitate wound, tissue, or bone repair. Additionally, such compositions can be administered in combination with an additional therapy.

### A. Carriers and Excipients

Pharmaceutically acceptable carriers or excipients may be used to deliver embodiments as described herein. Excipient refers to an inert substance used as a diluent or vehicle for a therapeutic agent. Pharmaceutically acceptable carriers are used, in general, with a compound (eg. peptide of the disclosure) so as to make the compound useful for a therapy or as a product. In general, for any substance, a carrier is a material that is combined with the substance for delivery to an animal. Conventional pharmaceutical carriers, aqueous, powder or oily bases, thickeners and the like may be necessary or desirable. In some cases the carrier is essential for delivery, e.g., to solubilize an insoluble compound for liquid delivery; a buffer for control of the pH of the substance to preserve its activity; or a diluent to prevent loss of the substance in the storage vessel. In other cases, however, the carrier is for convenience, e.g., a liquid for more convenient administration. Pharmaceutically acceptable salts of the compounds described herein may be synthesized according to methods known to those skilled in the arts. Thus a pharmaceutically acceptable compositions are highly purified to be free of contaminants, are sterile, biocompatible and not toxic, and further may include a carrier, salt, or excipient suited to administration to a patient. In the case of water as the carrier, the water is highly purified and processed to be free of contaminants, e.g., endotoxins.

The compounds described herein may be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. Thus the deliverable compound may be made in a form suitable for oral, rectal, topical, intravenous injection, intra-articular injection, intradermal, intramuscular, and/or parenteral administration. Carriers include solids or liquids, and the type of carrier is chosen based on the type of administration being used. Suitable binders, lubricants, disintegrating agents, coloring agents, flavoring agents, flow-inducing agents, and melting agents may be included as carriers, e.g., for pills. For instance, an active component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. The compounds can be administered orally in solid dosage forms, such as capsules, tablets, and powders, or in liquid dosage forms, such as elixirs, syrups, and suspensions. The active compounds can also be administered parentally, in sterile liquid dosage forms. Buffers for achieving a physiological pH or osmolarity may also be used.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

### B. Dosage

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8. 1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein of an agent of the disclosure (e.g. growth factor, cytokine, peptide, polypeptide, functional moiety, etc. . . . ).

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of the condition have disappeared or been reduced or after 6, 12, 18, or 24 hours or 1, 2, 3, 4, or 5 days after symptoms of an infection have disappeared or been reduced.

### V. Therapeutic Methods

The methods and compositions of the disclosure may be used to assist in the healing of normal wounds, including those resulting from accidents, surgery or failure of healing of a surgical wound (e.g., a dehiscent wound). The compositions and methods of the disclosure will accelerate wound healing, reduce scarring and ultimately promote repair, regeneration and restoration of structure and function in all tissues.

The methods and compositions of the disclosure can be used to treat external wounds caused by, but not limited to scrapes, cuts, lacerated wounds, bite wounds, bullet wounds, stab wounds, burn wounds, sun burns, chemical burns, surgical wounds, bed sores, radiation injuries, all kinds of acute and chronic wounds, wounds or lesions created by cosmetic skin procedures and also ameliorate the effects of skin aging. The methods and compositions of the disclosure may accelerate wound healing in all kinds of external wounds and improve the cosmetic appearance of wounded areas, and skin subject to aging and disease. The composition, peptide, polypeptide, implant, molecular complex, scaffold, or matrix of the disclosure may be provided directly, as a pre-treatment, as a pre-conditioning, coincident with injury, pre-injury, or post-injury. The composition may be used to treat internal injury caused by, but not limited to, disease, surgery, gunshots, stabbing, accidents, infarcts, ischemic injuries, to organs and tissues including but not limited to heart, bone, brain, spinal cord, retina, peripheral nerves and other tissues and organs commonly subject to acute and chronic injury, disease, congenital and developmental malformation and aging processes. Injury to internal organs causes a fibrotic response, which leads to loss of structure and function in organ systems.

Regenerative processes aided by the compositions and polypeptides of the disclosure may include, but are not limited to internal and external injury, regeneration of tissues, organs, or other body parts, healing and restoration of function following vascular occlusion and ischemia, brain stroke, myocardial infarction, spinal cord damage, brain damage, peripheral nerve damage, ocular damage (e.g., to corneal tissue), bone damage and other insults to tissues causing destruction, damage or otherwise resulting from, but not limited to, injury, surgery, cancer, congenital and developmental malformation, and diseases causing progressive loss of tissue structure and function, including but not limited to diabetes, bacterial, viral and prion-associated diseases, Alzheimer's disease, Parkinson's disease, AIDs and other genetically determined, environmentally determined or idiopathic disease processes causing loss of tissue/organ/body part structure and function. In addition, the compositions described herein can be administered with drugs or other compounds promoting tissue and cellular regeneration including, but not limited to, trophic factors in processes including, but not limited to, brain, retina, spinal cord and peripheral nervous system regeneration (e.g., NGFs, FGFs, Neurtrophins, Neuregulins, Endothelins, GDNFs, BDNF. BMPs, TGFs, Wnts), as well as pre-conditioning factors or stimuli e.g., hypoxia, norepinephrine, bradykinin, anesthetics, nitrate, ethanol, Alda-1, ALDH2 antagonists, PKC-epsilon agonists, exogenous ligands that activate opioid receptors (DPDPE, deltorphin II, methadone, SNC-80, BW373U86, DPI-287, DPI-3290) delivered in a prospective pre-treatment prior to a surgery of other procedure disrupting tissue in a subject.

Embodiments of the disclosure further include the use of the peptides, compositions, polypeptides, of the disclosure to aid in the healing of pathological wounds. Diabetic wounds are examples of difficult to heal wound can include, for example, a wound that is often characterized by slower than normal re-epithelialization/closure inflammatory phase and delayed formation and remodeling of extracellular matrix.

The present disclosure can also assist in the healing of chronic wounds or wounds that do not heal. Wounds that have not healed within three months, for example, are said to be chronic. Chronic wounds include, diabetic, diabetic foot, ischemic, venous, venous stasis, arterial, pressure, vasculitic, infectious, decubitis, burn, trauma-induced, gangrenous and mixed ulcers. Chronic wounds include, wounds that are characterized by and/or chronic inflammation, deficient and overprofuse granulation tissue differentiation and failure of re-epithelialization and wound closure and longer repair times. Chronic wounds can include ocular ulcers, including corneal ulcers. Use of the disclosed embodiments in would healing and tissue regeneration would include in humans and agricultural, sports and pet animals.

The subject may be one that has, has been diagnosed with, or has been determined to be at high risk for a cancer. The cancer may include or exclude tumors of all types, locations, sizes, and characteristics. The cancer may comprise or exclude a solid tumor. The cancer can comprise or exclude pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, lung cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma, squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### VI. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Flt3L Variants for Use in Healing of Non-Healing Wounds

Many conditions can cause the dysfunctional signaling environment that impairs wound healing, one of the most well studied is the diabetic disease state. In the diabetic disease state, chronic wounds have been shown to have poorly regulated inflammatory signals which has been linked to non-productive inflammation and the subsequent stagnation of the wound healing process (13). Dendritic cells (DC) are one cellular population, that have been found to be reduced in frequency between a healthy, healing wound and a diabetic, non-healing wound (14). DCs serve as an intermediary between the innate and adaptive immune system and carry out many functions within both arms of immunity (15). One purpose DCs serve is that the maintenance of homeostasis through the clearance of cellular debris, termed efferocytosis (16,17). Within the context of the diabetes, it has been shown that there is a higher burden of apoptotic cells in tissue of a diabetic wound when compared to tissue of a healthy wound (18). Using compositions comprising Flt3L polypeptides, the inventors have been able to increase the frequency of dendritic cells within the tissue of a diabetic wound and subsequently improve wound healing in a type 2 mouse model of diabetes.

Radiation therapy, a primary treatment of head and neck cancer, can cause the formation of lesions in the oral cavity, sometimes so severe that treatment must be interrupted leaving the cancer unchecked; this phenomenon is referred to as radiation induced oral mucositis (RIOM) (20). Radiation therapy not only causes these lesions to form, but also disrupts the highly regulated sequence required to heal them, creating another subset of non-healing wounds. Radiation therapy creates a substantial amount of cellular death and debris, specifically in cells of the hematopoietic system (21,22). The inventors hypothesize that, through the induction of more dendritic cells, the debris present in the oral lesions caused by radiation therapy can be cleared, thus breaking the pro-inflammatory cycle and allowing for healing of the mucosa. They further postulate that Flt3L will not have a negative effect on tumor control, as it has been shown previously to enhance tumor control in combination with radiotherapy and an immunostimulatory adjuvant as well as synergy with checkpoint blockade in human clinical trials (26,27).

Flt3L is a soluble, focused signal that can expand the dendritic cell compartment. While it has been studied and utilized in many contexts, it has not been studied in the context of non-healing wounds. Through engineering strategies the inventors were able to reduce the necessary dose to improve wound healing in the type 2 diabetic mouse model by 20-fold. The figures demonstrate that Flt3L heals chronic wounds in a type 2 diabetic mouse model (FIG. 1) and that Flt3L induces increase in DCs within the wound of type 2 diabetic mouse (FIG. 2).

### Example 2: Flt3L in Wound Healing

Wound healing is an important biological process that allows organisms to maintain homeostasis; without the potential to regenerate physiological barriers, we would be left vulnerable to infection. Chronic, non-healing wounds have reached epidemic proportions and have a sustainable impact on both an individual's quality of life and on healthcare costs as a whole (1-5). There are four main phases of wound healing: hemostasis, inflammation, proliferation, and remodeling, which direct the regeneration of damaged tissue. These phases overlap and require tightly regulated and coordinated signals (6, 7), which often come in the form of chemokines (directing immune cells to the damaged tissue) and cytokines—which inform cells present in the milieu what purpose they should serve (8-10). Chronic wounds have been shown to have both dysregulated temporal control and absolute expression of these soluble chemokine and cytokine signals, leading to the stagnation of healing (10-12).

Many conditions can cause the dysfunctional signaling environment that impairs wound healing, one of the most well studied is the diabetic disease state. In the diabetic disease state, chronic wounds have been shown to have poorly regulated inflammatory signals which has been linked to non-productive inflammation and the subsequent stagnation of the wound healing process (13). Dendritic cells (DC) are one cellular population, that have been found to be reduced in frequency between a healthy, healing wound and a diabetic, non-healing wound (14). DCs serve as an intermediary between the innate and adaptive immune system and carry out many functions within both arms of immunity (15). One purpose DCs serve is that the maintenance of homeostasis through the clearance of cellular debris, termed efferocytosis (16, 17). Within the context of the diabetes, it has been shown that there is a higher burden of apoptotic cells in tissue of a diabetic wound when compared to tissue of a healthy wound (18). It has been demonstrated that healing of the diabetic wound can be improved by enhancing the efferocytotic capacity of DCs in the diabetic wound (19). The benefit of dendritic cells in the chronic wound environment may be due to expression of both surface bound and soluble mediators which are capable of pushing the immune system towards a pro-resolution state. In a similar manner, through our work with Flt3L, we have been able to increase the frequency of dendritic cells within the tissue of a diabetic wound and subsequently improve wound healing in a type 2 mouse model of diabetes.

Radiation therapy, a primary treatment of head and neck cancer, can cause the formation of lesions in the oral cavity, sometimes so severe that treatment must interrupted leaving the cancer unchecked; this phenomenon is referred to as radiation induced oral mucositis (RIOM) (20). Radiation therapy not only causes these lesions to form, but also disrupts the highly regulated sequence required to heal them, creating another subset of non-healing wounds. Radiation therapy creates a substantial amount of cellular death and debris, specifically in cells of the hematopoietic system (21, 22). With the death of many immune cells that are tasked with clearing debris, dendritic cells among them, these lesions create an environment that is high in inflammatory signals that further potentiate cell damage and ultimately cell death, creating a feed-forward loop (23-25). We hypothesize that through the induction of more dendritic cells, the debris present in the oral lesions caused by radiation therapy can be cleared, thus breaking the pro-inflammatory cycle and allowing for healing of the mucosa. We further postulate that Flt3L will not have a negative effect on tumor control, as it has been shown previously to enhance tumor control in combination with radiotherapy and an immunostimulatory adjuvant as well as synergy with checkpoint blockade in human clinical trials (26, 27).

Flt3L is a soluble, focused signal that can expand the dendritic cell compartment. While it has been studied and utilized in many contexts, through our work we apply its benefits within the niche of treating non-healing wounds, something that has not been done previously. Additionally, through engineering strategies we were able to reduce the necessary dose to improve wound healing in the type 2 diabetic mouse model by 20-fold.

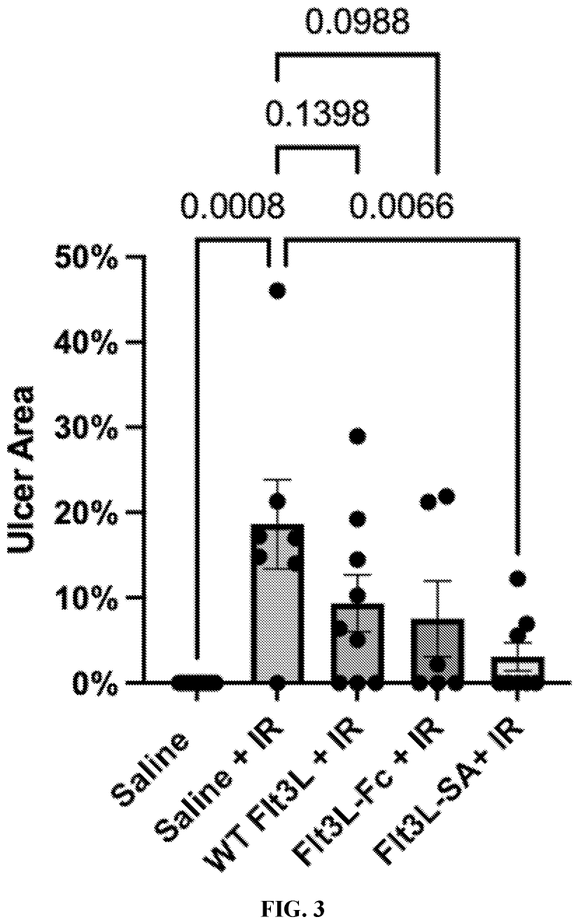

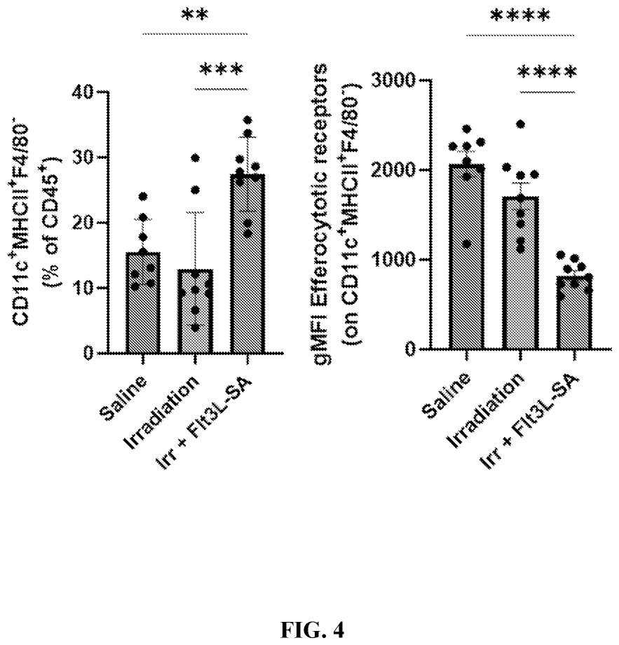

As shown in FIG. 1, Flt3L heals chronic wounds in a Type 2 Diabetic model. Flt3L, both wild type (WT) and an engineered construct (MSA) improves wound closure in a mouse model of type 2 diabetic (db/db) mice. WT Flt3L, at a dose of 10 μg, administered to the rear right hock immediately following wounding on day 0, improves wound closure in a mouse model of type 2 diabetes. Long-lived, engineered Flt3L (MSA-Flt3L) reduces therapeutic dose of WT Flt3L 20-fold. As shown in FIG. 2, Flt3L induces an increase in DCs within the wound of a Type 2 diabetic mouse. Both Flt3L variants demonstrate a significant increase in DC cellularity in the wound bed. As shown in FIG. 3, Flt3L-SA protects against the effects of radiation induced ulceration of the tongue in a statistically significant manner, and Flt3L-Fc shows near significant trends in protection. As shown in FIG. 4, Flt3L-SA increases DC cellularity in the tongue following irradiation. Decrease in efferocytic marker (Axl and Mertk) may be attributable to an increased efferocytic capacity of the DCs to clear wound debris in the irradiated tissue.

TABLE
Protein Sequences

| Protein | | SEQ ID |
|---|---|---|
| Construct | Protein Sequence | NO |
| murine | MTPDCYFSHSPISSNFKVKFRELTDHLLKDYPVTVAVNLQDEKHC | 58 |
| Flt3L | KALWSLFLAQRWIEQLKTVAGSKMQTLLEDVNTEIHFVTSCTFQP | |
| | LPECLRFVQTNISHLLKDTCTQLLALKPCIGKACQNFSRCLEVQCQ | |
| | PDSSTLLPPRSPIALEATELPEPRPRQ | |
| Human | MTQDCSFQHSPISSDFAVKIRELSDYLLQDYPVTVASNLQDEELCG | 59 |
| Flt3L | GLWRLVLAQRWMERLKTVAGSKMQGLLERVNTEIHFVTKCAFQ | |
| | PPPSCLRFVQTNISRLLQETSEQLVALKPWITRQNFSRCLELQCQPD | |
| | SSTLPPPWSPRPLEATAPTA | |
| Murine | EAHKSEIAHRYNDLGEQHFKGLVLIAFSQYLQKCSYDEHAKLVQE | 60 |
| Serum | VTDFAKTCVADESAANCDKSLHTLFGDKLCAIPNLRENYGELADC | |
| Albumin | CTKQEPERNECFLQHKDDNPSLPPFERPEAEAMCTSFKENPTTFMG | |
| | HYLHEVARRHPYFYAPELLYYAEQYNEILTQCCAEADKESCLTPK | |
| | LDGVKEKALVSSVRQRMKCSSMQKFGERAFKAWAVARLSQTFP | |
| | NADFAEITKLATDLTKVNKECCHGDLLECADDRAELAKYMCENQ | |
| | ATISSKLQTCCDKPLLKKAHCLSEVEHDTMPADLPAIAADFVEDQ | |
| | EVCKNYAEAKDVFLGTFLYEYSRRHPDYSVSLLLRLAKKYEATLE | |
| | KCCAEANPPACYGTVLAEFQPLVEEPKNLVKTNCDLYEKLGEYGF | |
| | QNAILVRYTQKAPQVSTPTLVEAARNLGRVGTKCCTLPEDQRLPC | |
| | VEDYLSAILNRVCLLHEKTPVSEHVTKCCSGSLVERRPCFSALTVD | |
| | ETYVPKEFKAETFTFHSDICTLPEKEKQIKKQTALAELVKHKPKAT | |
| | AEQLKTVMDDFAQFLDTCCKAADKDTCFSTEGPNLVTRCKDALA | |
| Human | MKWVTFISLLFLFSSAYSRGVFRRDAHKSEVAHRFKDLGEENFKA | 64 |
| Serum | LVLIAFAQYLQQCPFEDHVKLVNEVTEFAKTCVADESAENCDKSL | |
| Albumin | HTLFGDKLCTVATLRETYGEMADCCAKQEPERNECFLQHKDDNP | |
| | NLPRLVRPEVDVMCTAFHDNEETFLKKYLYEIARRHPYFYAPELL | |
| | FFAKRYKAAFTECCQAADKAACLLPKLDELRDEGKASSAKQRLK | |
| | CASLQKFGERAFKAWAVARLSQRFPKAEFAEVSKLVTDLTKVHT | |
| | ECCHGDLLECADDRADLAKYICENQDSISSKLKECCEKPLLEKSHC | |
| | IAEVENDEMPADLPSLAADFVESKDVCKNYAEAKDVFLGMFLYE | |
| | YARRHPDYSVVLLLRLAKTYETTLEKCCAAADPHECYAKVFDEF | |
| | KPLVEEPQNLIKQNCELFEQLGEYKFQNALLVRYTKKVPQVSTPT | |
| | LVEVSRNLGKVGSKCCKHPEAKRMPCAEDYLSVVLNQLCVLHEK | |
| | TPVSDRVTKCCTESLVNRRPCFSALEVDETYVPKEFNAETFTFHAD | |
| | ICTLSEKERQIKKQTALVELVKHKPKATKEQLKAVMDDFAAFVEK | |
| | CCKADDKETCFAEEGKKLVAASQAALGL | |
| Murine | AKTTPPSVYPLAPGSAAQTNSMVTLGCLVKGYFPEPVTVTWNSGS | 62 |
| IgG1 Fc | LSSGVHTFPAVLQSDLYTLSSSVTVPSSPRPSETVTCNVAHPASSTK | |
| | VDKKIVPRDCGCKPCICTVPEVSSVFIFPPKPKDVLTITLTPKVTCV | |
| | VVDISKDDPEVQFSWFVDDVEVHTAQTQPREEQFNSTFRSVSELPI | |
| | MHQDWLNGKEFKCRVNSAAFPAPIEKTISKTKGRPKAPQVYTIPPP | |
| | KEQMAKDKVSLTCMITDFFPEDITVEWQWNGQPAENYKNTQPIM | |
| | NTNGSYFVYSKLNVQKSNWEAGNTFTCSVLHEGLHNHHTEKSLS | |
| | HSPGK | |
| Human | ASTKGPSVFPLAPSSKSTSGGTAALGCLVKDYFPEPVTVSWNSGA | 63 |
| IgG1 Fc | LTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMIS | |
| | RTPEVTCVVVDVSHEDPEVKFNWYVDGVEVHNAKTKPREEQYNS | |
| | TYRVVSVLTVLHQDWLNGKEYKCKVSNKALPAPIEKTISKAKGQP | |
| | REPQVYTLPPSRDELTKNQVSLTCLVKGFYPSDIAVEWESNGQPE | |
| | NNYKTTPPVLDSDGSFFLYSKLTVDKSRWQQGNVFSCSVMHEAL | |
| | HNHYTQKSLSLSPGK | |

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

### REFERENCES

The following references, to the extent that they provide exemplary procedural or other details supplementary to those set forth herein, are specifically incorporated herein by reference.
1. Eming S A, Martin P, Tomic-Canic M. Wound repair and regeneration: mechanisms, signaling, and translation. Sci Transl Med. 2014 Dec. 3; 6(265):265sr6.
2 Las Heras K, Igartua M, Santos-Vizcaino E, Hernandez R M. Chronic wounds: Current status, available strategies and emerging therapeutic solutions. J Control Release. 2020; 328:532-550.
3 Olsson M, Järbrink K, Divakar U, Bajpai R, Upton Z, Schmidtchen A, Car J. The humanistic and economic burden of chronic wounds: A systematic review. Wound Repair Regen. 2019; 27(1):114-125.
4. Martinengo L, Olsson M, Bajpai R, Soljak M, Upton Z, Schmidtchen A, Car J, Järbrink K. Prevalence of chronic wounds in the general population: systematic review and meta-analysis of observational studies. Ann Epidemiol. 2019; 29:8-15.
5 Kinmond K, McGee P, Gough S, Ashford R. “Loss of self”: a psychosocial study of the quality of life of adults with diabetic foot ulceration. J Tissue Viability. 2003 January; 13(1):6-8, 10, 12 passim.
6. Landen N X, Li D, Ståhle M. Transition from inflammation to proliferation: a critical step during wound healing. Cell Mol Life Sci. 2016; 73(20):3861-85.
7. Reinke J M, Sorg H. Wound repair and regeneration. Eur Surg Res. 2012; 49 (1): 35-43.
8 MacLeod A S, Mansbridge J N. The Innate Immune System in Acute and Chronic Wounds. Adv wound care. 2016 Feb. 1; 5(2):65-78.

Ridiandries A, Tan J T M, Bursill C A. The Role of Chemokines in Wound Healing. Int J Mol Sci. 2018 Oct. 18; 19 (10).
10. Fivenson D P, Faria D T, Nickoloff B J, Poverini P J, Kunkel S, Burdick M, Strieter R M. Chemokine and inflammatory cytokine changes during chronic wound healing. Wound Repair Regen. 1997; 5(4):310-22.
11. Falanga V. Growth factors and wound healing. J Dermatol Surg Oncol. 1993 August; 19(8):711-4.
12. Barrientos S, Stojadinovic O, Golinko M S, Brem H, Tomic-Canic M. Growth factors and cytokines in wound healing. Wound Repair Regen. 2008; 16(5):585-601.
13. Sawaya A P, Stone R C, Brooks S R, Pastar I, Jozic I, Hasneen K, O'Neill K, Mehdizadeh S, Head C R, Strbo N, Morasso M I, Tomic-Canic M. Deregulated immune cell recruitment orchestrated by FOXM1 impairs human diabetic wound healing. Nat Commun. 2020; 11(1):4678.
14. Joshi N, Pohlmeier L, Ben-Yehuda Greenwald M, Haertel E, Hiebert P, Kopf M, Werner S. Comprehensive characterization of myeloid cells during wound healing in healthy and healing impaired diabetic mice. Eur J Immunol. 2020; 50(9):1335-1349.
15. Banchereau J, Steinman R M. Dendritic cells and the control of immunity. Nature. 1998 Mar. 19; 392(6673):245-52.
16. Hochreiter-Hufford A, Ravichandran K S. Clearing the dead: apoptotic cell sensing, recognition, engulfment, and digestion. Cold Spring Harb Perspect Biol. 2013 Jan. 1; 5(1):a008748.
17. McCubbrey A L, Curtis J L. Efferocytosis and lung disease. Chest. 2013 June; 143(6):1750-1757.
18. Khanna S, Biswas S, Shang Y, Collard E, Azad A, Kauh C, Bhasker V, Gordillo G M, Sen C K, Roy S. Macrophage dysfunction impairs resolution of inflammation in the wounds of diabetic mice. PLOS One. 2010 Mar. 4; 5(3):e9539.
19. Maschalidi S, Mehrotra P, Keçeli B N, De Cleene H K L, Lecomte K, Van der Cruyssen R, Janssen P, Pinney J, van Loo G, Elewaut D, Massie A, Hoste E, Ravichandran K S. Targeting SLC7A11 improves efferocytosis by dendritic cells and wound healing in diabetes. Nature. 2022; 606(7915):776-784.
20. Maria O M, Eliopoulos N, Muanza T. Radiation-Induced Oral Mucositis. Front Oncol. 2017; 7:89.
21. Eriksson D, Stigbrand T. Radiation-induced cell death mechanisms. Tumour Biol. 2010 August; 31(4):363-72.
22. Lauber K, Ernst A, Orth M, Herrmann M, Belka C. Dying cell clearance and its impact on the outcome of tumor radiotherapy. Front Oncol. 2012; 2:116.
23. Criswell T, Leskov K, Miyamoto S, Luo G, Boothman D A. Transcription factors activated in mammalian cells after clinically relevant doses of ionizing radiation. Oncogene. 2003 Sep. 1; 22(37):5813-27.
24. Liu S, Zhao Q, Zheng Z, Liu Z, Meng L, Dong L, Jiang X. Status of Treatment and Prophylaxis for Radiation-Induced Oral Mucositis in Patients With Head and Neck Cancer. Front Oncol. 2021; 11:642575.
25. Sonis S T. Mucositis: The impact, biology and therapeutic opportunities of oral mucositis. Oral Oncol. 2009 December; 45(12):1015-20.
26. Salmon H, Idoyaga J, Rahman A, Leboeuf M, Remark R, Jordan S, Casanova-Acebes M, Khudoynazarova M, Agudo J, Tung N, Chakarov S, Rivera C, Hogstad B, Bosenberg M, Hashimoto D, Gnjatic S, Bhardwaj N, Palucka A K, Brown B D, Brody J, Ginhoux F, Merad M. Expansion and Activation of CD103(+) Dendritic Cell Progenitors at the Tumor Site Enhances Tumor Responses to Therapeutic PD-L1 and BRAF Inhibition. Immunity. 2016 Apr. 19; 44(4):924-38.
27. Hammerich L, Marron T U, Upadhyay R, Svensson-Arvelund J, Dhainaut M, Hussein S, Zhan Y, Ostrowski D, Yellin M, Marsh H, Salazar A M, Rahman A H, Brown B D, Merad M, Brody J D. Systemic clinical tumor regressions and potentiation of PD1 blockade with in situ vaccination. Nat Med. 2019; 25(5):814-824.

1. A method for regenerating tissue, for preventing wound formation, or for treating wounds in a subject, the method comprising administering a composition comprising a fms related tyrosine kinase 3 ligand (Flt3L) polypeptide to the subject.

2. The method of claim 1, wherein the composition is administered intravenously or subcutaneously.

3. The method of claim 1 or 2, wherein the subject has cancer.

4. The method of claim 3, wherein the subject has head and neck cancer.

5. The method of any one of claims 1-4, wherein the subject has been administered or has been prescribed radiation therapy and/or chemotherapy.

6. The method of claim 5, wherein the subject has been prescribed radiation therapy and/or chemotherapy, and wherein the composition is administered prior to the therapy, after the therapy, or concurrently with the therapy.

7. The method of claim 6, wherein the composition is administered at a time period of within 24 hours of the therapy and either before or after the therapy.

8. The method of any one of claims 1-7, wherein the wound comprises skin atrophy, soft tissue fibrosis, desquamation, epithelial ulceration, fistula formation, and/or vessel rupture.

9. The method of any one of claims 1-8, wherein the subject has diabetes.

10. The method of claim 9, wherein the diabetes comprises type 1 or type 2 diabetes.

11. The method of any one of claims 1-10, wherein the method comprises treating a wound.

12. The method of claim 11, wherein the method comprises treating radiation-induced oral mucositis (RIOM).

13. The method of claim 11 or 12, wherein the wound comprises a chronic wound or the subject comprises a subject with chronic wounds.

14. The method of any one of claims 1-13, wherein the method provides for the increase in the frequence of dendritic cells within the tissue of the wound.

15. The method of any one of claims 1-14, wherein the wound comprises an infectious wound, ischemic wound, radiation poisoning wound, surgical wound, or ulcer.

16. The method of claim 15, wherein the wound comprises an ulcer and wherein the ulcer comprises a arterial ulcer, venous ulcer, diabetic ulcer, or pressure ulcer.

17. The method of any one of claims 1-16, wherein the method excludes acute wounds.

18. The method of any one of claims 1-17, wherein the method excludes burn wounds.

19. The method of any one of claims 1-18, wherein the subject has undergone or will undergo wound debridement.

20. The method of claim 19, wherein the composition is administered before, after, or concurrently with debridement of the wound.

21. The method of claim 20, wherein the composition is administered within 24 hours of wound debridement.

22. The method of any one of claims 1-21, wherein the composition is administered in two doses.

23. The method of any one of claims 1-22, wherein the subject has been administered an additional therapy or has been prescribed an additional therapy.

24. The method of any one of claims 1-23, wherein the subject has not been administered an additional therapy.

25. The method of claim 23 or 24, wherein the additional therapy comprises an immunotherapy and/or an immune agonist.

26. The method of any one of claim 1-25, wherein the FLT3L polypeptide comprises a fusion protein comprising: a FLT3L polypeptide linked to a serum protein.

27. The method of claim 26, wherein the serum protein comprises albumin.

28. The method of claim 27, wherein the albumin comprises human serum albumin.

29. The method of any one of claims 26-29, wherein the serum protein comprises the amino acid sequence of SEQ ID NO:60, 61 or 64, or an amino acid sequence having at least 80% sequence identity to SEQ ID NO:60, 61 or 64.

30. The method of any one of claims 1-29, wherein the FLT3L polypeptide comprises the amino acid sequence of one of SEQ ID NOs:40-50, 58 or 59 or an amino acid sequence having at least 80% sequence identity to one of SEQ ID NOs:40-50, 58 or 59.

31. The method of any one of claim 1-30, wherein the FLT3L polypeptide comprises a fusion protein comprising: a FLT3L extracellular domain operably linked to an immunoglobulin fragment crystallizable region (Fc region).

32. The method of claim 31, wherein at least 5 amino acids are truncated from the C-terminus of the FLT3L extracellular domain; and/or the Fc region does not comprise a hinge region.

33. The method of claim 31 or 32, wherein the FLT3L extracellular domain is a human FLT3L extracellular domain or derived from a human FLT3L extracellular domain.

34. The method of any one of claims 31-33, wherein the fusion protein is capable of binding to human FLT3.

35. The method of any one of claims 31-34, wherein the FLT3L extracellular domain is from FLT3L isoform 1.

36. The method of any one of claims 31-34, wherein the FLT3L extracellular domain is from FLT3L isoform 2.

37. The method of any one of claims 31-36, wherein the FLT3L extracellular domain does not comprise the amino acid sequence PTAPQ.

38. The method of any one of claims 31-37, wherein at least 6, 7, 8, 9, 10, 11, 12, 13, 14 or 15 amino acids are truncated from the C-terminus of the FLT3L extracellular domain.

39. The method of any one of claims 31-38, wherein the FLT3L extracellular domain does not comprise the amino acid sequence APTAPQ (SEQ ID NO:29), TAPTAPQ (SEQ ID NO:30), ATAPTAPQ (SEQ ID NO:31), EATAPTAPQ (SEQ ID NO:32), or LEATAPTAPQ (SEQ ID NO:33).

40. The method of any one of claims 31-39, wherein the FLT3L extracellular domain does not comprise the amino acid sequence PTAPQPP (SEQ ID NO:34), APTAPQPP (SEQ ID NO:35), TAPTAPQPP (SEQ ID NO:36), ATAPTAPQPP (SEQ ID NO:37), EATAPTAPQPP (SEQ ID NO:38), or LEATAPTAPQPP (SEQ ID NO:39).

41. The method of any one of claims 31-40, wherein the FLT3L extracellular domain comprises an N-terminal signal peptide.

42. The method of any one of claims 31-41, wherein the FLT3L extracellular domain comprises amino acid substitutions at one or more of the following amino acid positions: H8Y, K84E, N100, S102, N123 and S125, wherein the amino acid residue positions are with reference to SEQ ID NOs:1-18, 21-27 or 40-50.

43. The method of any one of claims 31-42, wherein the FLT3L extracellular domain comprises one or more of the following amino acid substitutions: H8Y, K84E, S102A, and/or S125A; wherein the amino acid residue positions are with reference to SEQ ID NOs:1-18, 21-27 or 40-50.

44. The method of any one of claims 31-43, wherein one or both of serine residues at positions 102 and 125 are substituted to alanine, wherein the amino acid residue positions are with reference to SEQ ID NOs:1-18, 21-27 or 40-50.

45. The method of any one of claims 31-44, wherein the Fc region is from a human IgG1, IgG2, IgG3 or IgG4.

46. The method of any one of claims 31-45, wherein the Fc region comprises a human IgG1 isotype and comprises one or more amino acid substitutions in the Fc region at a residue position selected from the group consisting of: N297A, N297G, N297Q, N297G, D265A, L234A, L235A, C226S, C229S, P238S, E233P, L234V, P238A, A327Q, A327G, P329A, P329G, K322A, L234F, L235E, P331S, T394D, A330L, M252Y, S254T, T256E, M428L, N434S, T366W, T366S, L368A, Y407V, and any combination thereof, wherein the numbering of the residues is according to EU numbering.

47. The method of claim 46, wherein the Fc region comprises a human IgG1 isotype and comprises one or more amino acid substitutions in the Fc region at a residue position selected from the group consisting of: L234A, L234V, L234F, L235A, L235E, P331S, and any combination thereof, wherein the numbering of the residues is according to EU numbering.

48. The method of any one of claims 31-45, wherein the Fc region comprises a human IgG4 isotype and comprises one or more amino acid substitutions in the Fc region at a residue position selected from the group consisting of: E233P, F234V, F234A, L235A, G237A, E318A, S228P, L235E, T394D, M252Y, S254T, T256E, N297A, N297G, N297Q, T366W, T366S, L368A, Y407V, M428L, N434S, and any combination thereof, wherein the numbering of the residues is according to EU numbering.

49. The method of claim 48, wherein the Fc region comprises a human IgG4 isotype and comprises one or more amino acid substitutions in the Fc region at a residue position selected from the group consisting of: F234V, F234A, L235A, L235E, S228P, and any combination thereof, wherein the numbering of the residues is according to EU numbering.

50. The method of any one of claims 31-49, wherein the Fc region comprises the following amino acids at the indicated positions (EU index numbering): Tyrosine at position 252, threonine at position 254 and glutamic acid at position 256 (YTE); or Leucine at position 428 and serine at position 434 (LS).

51. The method of any one of claims 31-50, wherein the FLT3L extracellular domain comprises the amino acid sequence of one of SEQ ID NOs:40-50, or an amino acid sequence having at least 80% sequence identity to one of SEQ ID NOs:40-50.

52. The method of any one of claims 31-51, wherein the Fc region comprises the amino acid sequence of one of SEQ ID NOs:51-55, 62, and 63, or an amino acid sequence having at least 80% sequence identity to one of SEQ ID NOs:51-55, 62, and 63.

53. The method of any one of claims 31-52, wherein the fusion protein comprises the amino acid sequence of one of SEQ ID NOs:1-27, or an amino acid sequence having at least 80% sequence identity to one of SEQ ID NOs:1-27.

54. The method of any one of claims 31-53, wherein the Fc region is from a human IgG1 and does not comprise a hinge region.

55. The method of claim 54, wherein the C-terminus of the FLT3L extracellular domain is not truncated.

56. The method of any one of claims 31-55, wherein the Fc region is derived from a human IgG1 isotype and does not comprise a hinge region, e.g., does not the amino acid sequence EPKSCDKTHTCPPCP (SEQ ID NO:56) or EPKSCDKTHTCPPCPAPELL (SEQ ID NO:57).

57. The method of any one of claims 31-55, wherein the Fc region is from a human IgG4 and at least 5 amino acids are truncated from the C-terminus of the FLT3L extracellular domain.

58. The method of any one of claims 31-57, wherein the Fc region comprises a hinge region.

59. The method of any one of claims 31-58, wherein the Fc region is derived from a human IgG4 isotype and wherein at least 5 amino acids are truncated from the C-terminus of the FLT3L extracellular domain.

60. The method of claim 59, wherein the FLT3L extracellular domain does not comprise the amino acid sequence PTAPQ.


SEQUENCE LISTING (64 sequences)

SEQ ID NO: 1 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 364 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 386 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 386 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 381 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 359 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 376 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 364 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 386 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 386 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPLEATAPTAPQGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 381 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 359 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 376 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 376 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 376 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 389 aa, synthetic construct
TPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 20 - protein, 389 aa, synthetic construct
TPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTSTQLLALKPCIGKACQNFSRCLEVQCQPDSSTLLPPRSPIALEATELPEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 22 - protein, 369 aa, synthetic construct
TQDCSFQYSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTECAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 369 aa, synthetic construct
TQDCSFQYSPISSDFAVKIRELSDYLLQDYPVTVASNLQDEELCGGLWRLVLAQRWMERLKTVAGSKMQGLLERVNTEIHFVTECAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 381 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPLEATAESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 381 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDAAALPPPWSPRPLEATAESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 381 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDAAALPPPWAPRPLEATAEAKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 5 aa, synthetic construct
PTAPQ

SEQ ID NO: 29 - protein, 6 aa, synthetic construct
APTAPQ

SEQ ID NO: 30 - protein, 7 aa, synthetic construct
TAPTAPQ

SEQ ID NO: 31 - protein, 8 aa, synthetic construct
ATAPTAPQ

SEQ ID NO: 32 - protein, 9 aa, synthetic construct
EATAPTAPQ

SEQ ID NO: 33 - protein, 10 aa, synthetic construct
LEATAPTAPQ

SEQ ID NO: 34 - protein, 7 aa, synthetic construct
PTAPQPP

SEQ ID NO: 35 - protein, 8 aa, synthetic construct
APTAPQPP

SEQ ID NO: 36 - protein, 9 aa, synthetic construct
TAPTAPQPP

SEQ ID NO: 37 - protein, 10 aa, synthetic construct
ATAPTAPQPP

SEQ ID NO: 38 - protein, 11 aa, synthetic construct
EATAPTAPQPP

SEQ ID NO: 39 - protein, 12 aa, synthetic construct
LEATAPTAPQPP

SEQ ID NO: 40 - protein, 147 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRP

SEQ ID NO: 41 - protein, 148 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPL

SEQ ID NO: 42 - protein, 149 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLE

SEQ ID NO: 43 - protein, 150 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEA

SEQ ID NO: 44 - protein, 151 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEAT

SEQ ID NO: 45 - protein, 152 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATA

SEQ ID NO: 46 - protein, 153 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAP

SEQ ID NO: 47 - protein, 154 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPT

SEQ ID NO: 48 - protein, 155 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTA

SEQ ID NO: 49 - protein, 156 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAP

SEQ ID NO: 50 - protein, 157 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQ

SEQ ID NO: 51 - protein, 212 aa, synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 212 aa, synthetic construct
GGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 229 aa, synthetic construct
ESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 15 aa, synthetic construct
EPKSCDKTHTCPPCP

SEQ ID NO: 57 - protein, 20 aa, synthetic construct
EPKSCDKTHTCPPCPAPELL

SEQ ID NO: 58 - protein, 163 aa, synthetic construct
MTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQ

SEQ ID NO: 59 - protein, 156 aa, synthetic construct
MTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTA

SEQ ID NO: 60 - protein, 584 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 61 - protein, 609 aa, synthetic construct [sequence omitted]

SEQ ID NO: 62 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 63 - protein, 330 aa, synthetic construct [sequence omitted]

SEQ ID NO: 64 - protein, 609 aa, synthetic construct [sequence omitted]